(19) United States
(12) Patent Application Publication
(10) Pub. No.: US 2019/0133500 A1
(43) Pub. Date: May 9, 2019

(54) WEARABLE ASSAY SYSTEM AND METHOD OF USE

(71) Applicant: Intel Corporation, Santa Clara, CA (US)

(72) Inventor: Amar S Basu, (US)

(21) Appl. No.: 15/571,660

(22) PCT Filed: Dec. 8, 2016
(86) PCT No.: PCT/US2016/065671

§ 371 Date: Nov. 3, 2017

(51) Int. Cl. A61B 5/1477; A61B 5/145; A61B 5/1455

(52) U.S. Cl. CPC A61B 5/1477; A61B 5/1451; A61B 5/14517; A61B 5/14546; A61B 2560/0214; A61B 2560/0468; A61B 5/14556

(57) ABSTRACT

Various embodiments disclosed relate to a wearable assay system. The wearable assay system includes a first external surface adapted to contact a wearer and a void defined by a portion of the first external surface. A sample collection probe is positioned near the void and is attached to the first external surface. The sample collection probe is adapted to collect a biological sample from the wearer. An assay unit is adapted to receive the biological sample from the sample collection probe. An actuation system is adapted to position the assay unit in contact with the sample collection probe. A detection system is adapted to detect a property of the biological sample.

### BACKGROUND

Health conditions can be associated with the presence or concentration of certain biological molecules. For example, an increase in the concentration of a particular hormone can be correlated with a health condition. However, it is difficult to detect the presence or absence of biological molecules in real time. Normally a sample such as blood or interstitial fluid needs to be extracted and sent to a professional for analysis. This can be inconvenient and detrimental to one's health. There is therefore a need to increase the ease and efficacy of detecting the presence or absence of biological molecules in real or near-real time.

### BRIEF DESCRIPTION OF THE FIGURES

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a perspective view of a wearable assay system, according to an example of the present disclosure.

FIG. 2 is a sectional schematic view of the wearable assay system of FIG. 1.

FIG. 3 is a perspective view of an example of an assay unit in which the assay unit is a lateral flow strip

FIG. 4 is a perspective view of an assay unit in which the assay unit includes an array of lateral flow strips.

FIG. 5 is a perspective view of an assay unit in which the assay unit includes an array of lateral flow strips and a removable blocking layer.

FIG. 6 is a schematic view of an assay unit including an array of electrodes.

FIG. 7 is a circuit diagram showing a circuit for the assay unit of FIG. 6.

FIG. 8 is a circuit diagram showing an alternative circuit for the assay unit of FIG. 6.

FIG. 9 is a schematic sectional diagram showing an arrangement of the array of electrodes of FIG. 6.

FIG. 10 is a schematic sectional diagram showing an alternative arrangement of the array of electrodes of FIG. 6.

FIG. 11 is a schematic sectional diagram showing an additional alternative arrangement of the array of electrodes of FIG. 6.

FIG. 12 is a schematic diagram showing an assay unit that is a removable cartridge.

FIG. 13 is a schematic diagram showing an alternative configuration of the assay unit of FIG. 12.

FIG. 14 is a schematic diagram showing an additional alternative configuration of the assay unit of FIG. 12.

FIG. 15 is a perspective view showing a detection system of the wearable assay system of FIG. 1.

FIG. 16 is a schematic sectional view of a dynamo used in conjunction with the wearable assay system of FIG. 1.

FIG. 17 is a schematic view of the wearable assay system of FIG. 1 including a flexible membrane.

FIG. 18 is a sectional schematic view of the wearable assay system of FIG. 1 including a transpiration system.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the inventive subject matter, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

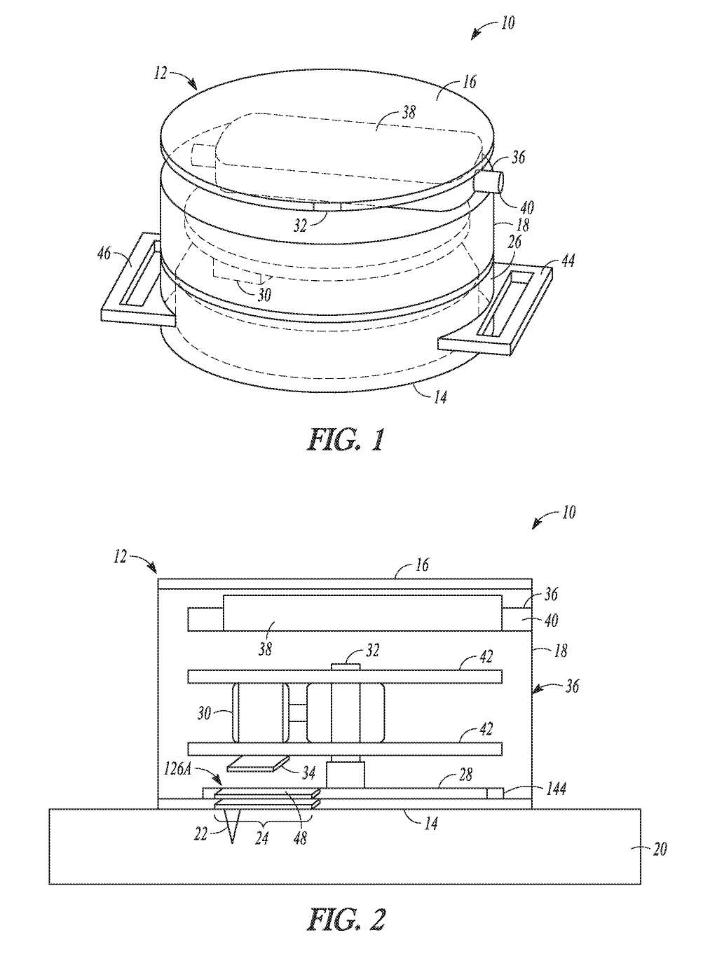

FIG. 1 is a perspective view of wearable assay system 10, according to an example of the present disclosure. FIG. 2 is a sectional schematic view of wearable assay system 10. FIGS. 1 and 2 show many of the same components and will be discussed concurrently

As illustrated, wearable assay system 10 includes housing 12. Housing 12 encases the components of wearable assay system 10 and is formed from bottom first external surface 14 and top second external surface 16, which are joined by side third external surface 18. Each surface has a corresponding internal surface defining the interior of housing 12.

Housing 12 is shown as having a generally cylindrical shape but may take on other shapes in further examples. For example, housing 12 may have a generally square or rectangular shape. Additionally, the shape of housing 12 may be nonuniform such that at least two surfaces have different shapes. For example, first external surface 14 may have a curved profile whereas second external surface 16 may have a generally planar surface. Such a configuration may be desirable in examples in which first external surface 14 is in direct contact with a wearer's skin 20. That is, a curved profile may improve user comfort or increase the contact points between first external surface 14 and the wearer's skin.

Housing 12 may be made of many different types of materials. For example, housing 12 may be made of a plastic formed from a polymer. For example, the polymer may include a polyurethane, polyethylene, polycarbonate, or copolymer thereof. Housing 12 may also be formed from a metal or alloy. Additionally, different sections of housing 12 (e.g., first external surface 14 and second external surface 16) may be made of different materials. The specific material(s) that form housing 12 depend on the desired durability, function, and aesthetics of wearable assay system 10. Additionally, the materials and overall size of housing 12 are selected to make wearable assay system 10 truly wearable. That is, the materials are selected to make wearable assay system 10 light enough to be comfortably worn. Additionally, the materials are selected from materials that will not cause an allergic reaction, or be toxic, to at least a substantial majority of wearers.

The interior of housing 12 includes many of the components of wearable assay system 10. As shown, sample collection probe 22 is attached to first external surface 14. More specifically, sample collection probe 22 is disposed within void 24, which is defined by first external surface 14. As discussed further herein, sample collection probe 22 may take on many different forms and is adapted to collect a biological sample from the wearer. Assay unit 26A is located proximate to sample collection probe 22. As described herein, assay unit 26A may be one of many different types of assay units adapted to perform any one of different types of assays upon receiving the biological sample from sample collection probe 22. Assay unit 26A is fixed to actuation system 28, which is adapted to position assay unit 26A in contact with sample collection probe 22. As described herein, actuation system 28 may take on many different forms. As shown, wearable assay system 10 is formed from a rotating disc in which assay unit 26A is placed. In various examples, a second assay unit 26A may be included and fixed to actuation system 28. In this example, actuation system 28 is adapted to selectively place assay unit 26A, second assay unit 26A, or any additional assay unit in contact with sample collection probe 22. Actuation system 28 may be driven in many different ways including by motor 30, which drives axle 32 connected to actuation system 28. As shown, motor 30 is a worm gear motor.

Wearable assay system 10 further includes detection system 34. As described herein, detection system 34 is adapted to detect a property of the biological sample. As shown in FIG. 1, detection system 34 is designed to be an optical detector. Detection system 34 may take on many different forms in other examples of wearable assay system 10.

Wearable assay system 10 also includes pumping system 36. Pumping system 36 may take on many different forms and may be either an active or a passive pumping system. As shown, pumping system 36 is an active system and includes pump 38, which is connected to valve 40 on third external surface 18.

Wearable assay system 10 further includes at least one printed circuit board 42. Printed circuit board 42 is used to control various systems in wearable assay system 10. For example, printed circuit board 42 may include drivers or microcontrollers that may be used to issue a command to actuation system 28 in order to place assay unit 26A, or any additional assay unit, in contact with sample collection probe 22. Printed circuit board 42 may also be used to activate detection system 34 and process data obtained from detection system 34. Printed circuit board 42 may also include antennas, radios, or other elements used for wireless communication. Additionally, printed circuit board 42 may be used to issue a command to pumping system 36 to activate or deactivate.

Printed circuit board 42 may be formed of many different types of components, including wires and silicon dies. Suitable silicon dies may include a memory die, a processing die, or any other suitable die to effect the function of wearable assay system 10. Printed circuit board 42 may be powered by a battery disposed within wearable assay system 10, or powered by an external power source.

Housing 12 may further include features such as an attachment member used to aid in attaching wearable assay system 10 to a wearer. For example, housing 12 may include first attachment member 44 and second attachment member 46. First attachment member 44 and second attachment member 46, as shown, are shaped to allow a first or second respective strap or band to be attached. The first and second bands may then be attached to each other in a manner similar to attaching a watch band (e.g., by a buckle or Velcro connection). The straps may be sized for attachment at any suitable location on the wearer. For example, the straps may be small enough to fit around a wearer's wrist or large enough to fit around a wearer's abdominal region or thigh.

In other examples, it may be desirable to configure wearable assay system 10 to be attached to the wearer in a manner akin to a skin patch. Accordingly, first attachment member 44 and second attachment member 46 may be replaced with an adhesive layer that is disposed on first external surface 14. The adhesive layer may be adapted to at least temporarily adhere the system to the wearer. The level of adhesion between the wearer and wearable assay system 10 may be a function of the adhesive used. Stronger adhesives may result in longer adhesion times. The adhesive layer may be applied directly to first external surface 14.

Additionally, first attachment member 44 and second attachment member 46 may form a flexible sheet attached to housing 12. The flexible sheet may be attached to the perimeter of first external surface 14, and the adhesive layer may be disposed on a surface of the flexible sheet that contacts the wearer. In other examples, the flexible sheet may be used as an interface to incorporate wearable assay system 10 into another wearable article. For example, the flexible sheet may be sewn into an article of clothing or into a cloth wrap or cast.

Once wearable assay system 10 is attached to the wearer, a biological sample may be collected from the wearer. The biological sample may be many different types of samples. For example, the biological sample may include a subdermal interstitial fluid. Interstitial fluid is a solution that bathes and surrounds the tissue cells of multicellular animals. It is the main component of extracellular fluid. Extracellular fluid includes plasma and transcellular fluid. The interstitial fluid is found in the interstices, which are spaces between cells. On average, humans include about 10 liters of interstitial fluid. Functionally, the interstitial fluid provides the cells of the body with nutrients and a means of waste removal.

The interstitial fluid may include several biomolecules of interest. For example, the interstitial fluid usually includes a water solvent having sugars, salts, fatty acids, amino acids, coenzymes, hormones, neurotransmitters, proteins, and enzymes, as well as waste products from the cell (e.g., metabolites) and white blood cells, electrolytes, heavy metals, and other items of biological interest dissolved therein. The exact composition of interstitial fluid depends upon the exchanges among the cells in the biological tissue and the blood. Accordingly, interstitial fluid has a different composition in different tissues and in different areas of the body. Different biomolecules, or concentrations of biomolecules, may be indicators for various health-related issues.

The weight percentage of the interstitial fluid that each component accounts for may vary. For example, proteins or enzymes may account for 1% (w/w) to about 5% (w/w), or less than about, equal to about, or greater than about 1.2% (w/w), 1.4, 1.6, 1.8, 2.0, 2.2, 2.4, 2.6, 2.8, 3.0, 3.2, 3.4, 3.6, 3.8, 4.0, 4.2, 4.4, 4.6, or 4.8% (w/w) of the subdermal interstitial fluid. Similarly, hormones may account for 1% (w/w) to about 5% (w/w), or less than about, equal to about, or greater than about 1.2% (w/w), 1.4, 1.6, 1.8, 2.0, 2.2, 2.4, 2.6, 2.8, 3.0, 3.2, 3.4, 3.6, 3.8, 4.0, 4.2, 4.4, 4.6, or 4.8% (w/w) of the subdermal interstitial fluid. Moreover, metabolites may account for 1% (w/w) to about 5% (w/w), or less than about, equal to about, or greater than about 1.2% (w/w). 1.4, 1.6, 1.8, 2.0, 2.2, 2.4, 2.6, 2.8, 3.0, 3.2, 3.4, 3.6, 3.8, 4.0, 4.2, 4.4, 4.6, or 4.8% (w/w) of the subdermal interstitial fluid. The weight percentage of each component of the interstitial fluid may be less than or greater than the ranges recited as well.

Wearable assay system 10 may also be used to perform assays on other bodily fluids. For example, wearable assay system 10 may be used to perform an assay on blood or sweat. Blood or sweat may include many of the same biomolecules as the subdermal interstitial fluid. The sweat is located on a layer of skin 20 of the wearer. For example, the sweat may include a protein or hormone. The protein may account for 1% (w/w) to about 5% (w/w), or less than about, equal to about, or greater than about 1.2% (w/w), 1.4, 1.6, 1.8, 2.0, 2.2, 2.4, 2.6, 2.8, 3.0, 3.2, 3.4, 3.6, 3.8, 4.0, 4.2, 4.4, 4.6, or 4.8% (w/w) of the sweat. Similarly, the hormone may account for 1% (w/w) to about 5% (w/w), or less than about, equal to about, or greater than about 1.2% (w/w), 1.4, 1.6, 1.8, 2.0, 2.2, 2.4, 2.6, 2.8, 3.0, 3.2, 3.4, 3.6, 3.8, 4.0, 4.2, 4.4, 4.6, or 4.8% (w/w) of the sweat.

The biological sample is initially collected by sample collection probe 22. As shown, sample collection probe 22 is a needle. In other examples, sample collection probe may be a suction pump or absorbent pad, amongst other suitable components. The needle is hollow, which allows the biological sample to pass therethrough. A length of the needle may be selected depending on the intended function of the needle. For example, the needle may be relatively short for applications in which the desired biological sample is located at or near the epithelial layer of the wearer's skin. Additionally, the needle may be longer for applications in which the desired biological sample is located at or near a deeper layer of the wearer's skin. The needle may be fixed in one position such that it extends beyond first external surface 14. This will lead to the needle puncturing the wearer's skin simultaneously with the attachment of wearable assay system 10 to the wearer.

In other examples, the needle may be adapted to be movable between a first position and a second position. The command to move between the first position and the second position may be issued by a die of printed circuit board 42. In the first position, the needle may be in an extended state such that a major portion of the needle is disposed outside of housing 12. In the second position, the needle may be in a retracted state such that a major portion of the needle is disposed within housing 12. This allows the needle to be in the retracted state when wearable assay system 10 is attached to the wearer. This may allow the needle to be stored until a predetermined time when sample collection is initiated. In some examples in which the needle is in the second position before wearable assay system 10 is attached to the wearer, a film may be placed over void 24. The film may help to prevent external contaminants from entering wearable assay system 10 and may help to prevent desterilizaiton of the needle. The needle may puncture the film as it moves through void 24 into the first position.

Once the needle has punctured the skin of the wearer, the biological sample may flow through the needle. In some examples, the biological sample may flow through or be pumped through the needle by capillary action. Capillary action (e.g., capillarity, capillary motion, or wicking) is generally understood to refer to the ability of a liquid to flow in narrow spaces without the assistance of, or even in opposition to, external forces such as gravity. The effect may be seen in the drawing up of liquids in a thin tube, in porous materials such as paper and plaster, in some non-porous materials such as sand and liquefied carbon fiber, or in a cell. Capillary action occurs because of intermolecular forces between the liquid and surrounding solid surfaces. If the diameter of the tube or pore is sufficiently small, then the combination of surface tension (which is caused by cohesion within the liquid) and adhesive forces between the liquid and container wall act to propel the liquid.

In other examples, sample collection probe 22 may be a wicking pad. The wicking pad is made of a porous material such as paper. The wicking pad is positioned on the wearer's skin and directly absorbs a biological sample (e.g., sweat). As in the example of the needle, the biological sample is wicked or transported through the wicking pad towards assay unit 26A. Although the wicking pad is not adapted to puncture skin like the needle, the wicking pad may still be used to collect a subdermal biological sample such as blood or an interstitial fluid. To accomplish this, the wicking pad may be used in conjunction with the needle. That is, the wicking pad may be disposed around the needle or adjacent to the needle to absorb subdermal biological samples that escape from the needle.

Assay unit 26A may be adapted to test for different biological properties of the particular fluid or biomolecule of interest. For example, assay unit 26A may be adapted to test for the presence of a particular biomolecule in the biological sample. Additionally, assay unit 26A may be adapted to determine the concentration of a particular biomolecule in the biological sample. That is, assay unit 26A may be able to specifically locate a biomolecule of interest in the biological sample and determine the concentration of that biomolecule in the biological sample. Various examples of assay unit 26A will be discussed herein.

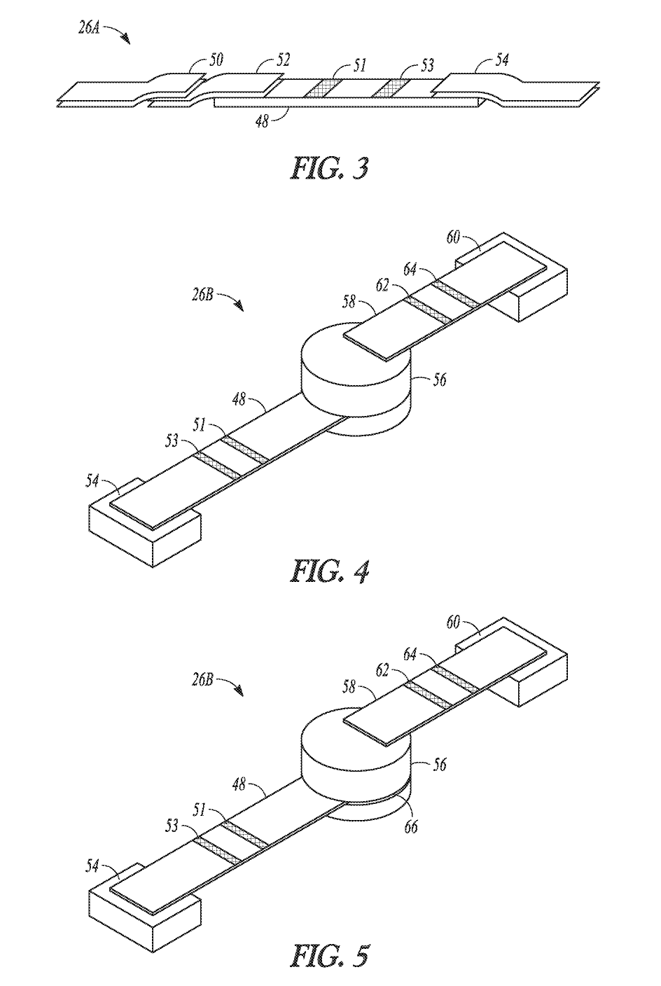

FIG. 3 is a perspective view of assay unit 26A in which assay unit 26A is a lateral flow strip. As shown, lateral flow strip 48 is rectangularly shaped and includes test line 51 and control line 53. Lateral flow strip 48 may be formed out of many different absorbent or porous materials. An example of a suitable material is nitrocellulose. Although one lateral flow strip 48 is shown, wearable assay system 10 may include multiple lateral flow strips arranged as an array on actuation system 28. An array can include anywhere from 2 to 100 members. FIG. 3 also shows absorbent sample pad 50, which is connected to absorbent conjugate pad 52. Absorbent conjugate pad 52 is attached to a first end of lateral flow strip 48. FIG. 3 also shows first wick pad 54, which is attached to a second end of lateral flow strip 48.

In operation of wearable assay system 10, the biological sample is initially collected by sample collection probe 22. The biological sample then wicks through sample collection probe 22 to sample pad 50. The biological sample wicks through sample pad 50 to conjugate pad 52. Conjugate pad 52 is preloaded with various nanoparticles that are adapted to attach to the biomolecule of interest. Suitable nanoparticles include antibodies that bind, for example, to a hormone or a protein. Typically, conjugate pad 52 will include more nanoparticles than necessary. Thus, when the nanoparticles join the flow of the biological sample, bound and free nanoparticles will be wicked to lateral flow strip 48. As the nanoparticles wick across lateral flow strip 48, nanoparticles bound to the biomolecule of interest attach to test line 51, whereas nanoparticles free of the biomolecule of interest attach to control line 53.

Aggregation of the nanoparticles on test line 51 and control line 53 may cause a change in the color and intensity of each line. The color change may be visible with the naked eye. Additionally, the color change may be detected by detection system 34, which may analyze and quantify the intensity and/or color of test line 51 and control line 53 and correlate that value to a concentration or presence of a particular biomolecule. Detection system 34 is discussed further herein. The color change may result from a chemical reaction of the nanoparticle with an exposed side group on test line 51 or control line 53. Additionally, the color change may result from emissions of fluorophores attached to the nanoparticles that emit light when stimulated.

FIG. 3 shows an example of assay unit 26A in which assay unit 26A is a single lateral flow strip. FIG. 4 shows an example of assay unit 26B which includes an array of lateral flow strips. In wearable assay system 10, assay unit 26B may be used in addition to and/or instead of assay unit 26A. As shown in FIG. 4, assay unit 26B further includes filter pad 56, second lateral flow strip 58, and second wick pad 60. Although only two lateral flow strips are shown, it is within the scope of this disclosure to include additional lateral flow strips in assay unit 26B. First lateral flow strip 48 and second lateral flow strip 58 are shown as oriented at a 180 degree angle with respect to each other, but any other suitable angle is possible. The driving consideration in the orientation of first lateral flow strip 48 and second lateral flow strip 58 is the orientation of each strip with respect to detection system 34. In some examples, it may be necessary to incorporate a second detection system in wearable assay system 10 to interact with second lateral flow strip 58. Alternatively, detection system 34 may be movable to selectively interact with first lateral flow strip 48 or second lateral flow strip 58.

Filter pad 56 is adapted to contact sample collection probe 22. First lateral flow strip 48 includes test line 51 and control line 53. Second lateral flow strip 58 includes second test line 62 and second control line 64. Filter pad 56 is formed from an absorbent material that wicks the biological sample towards first lateral flow strip 48. Filter pad 56 is preloaded with various nanoparticles that are adapted to attach to the biomolecule of interest. Suitable nanoparticles include antibodies that bind, for example, to a hormone or a protein. Typically, filter pad 56 will include more nanoparticles than necessary. Thus, when the nanoparticles join the flow of the biological sample, bound and free nanoparticles will be wicked to lateral flow strip 48. As the nanoparticles wick across lateral flow strip 48, nanoparticles bound to the biomolecule of interest attach to test line 51, whereas nanoparticles free of the biomolecule of interest attach to control line 53, as described above.

The biological sample continues to flow through first lateral flow strip 48 until first wick pad 54 is saturated. At this point the biological sample may no longer flow through first lateral flow strip 48. The biological sample then continues to flow through filter pad 56 to a second location where second lateral flow strip 58 is attached. The biological sample then continues to flow through second lateral flow strip 58 in a manner similar to that described with respect to first lateral flow strip 48.

Concentration of a different predetermined biological molecule may be detected by flow strip 58. In some examples of assay unit 26B, second test line 62 and second control line 64 may be adapted to attach to the same nanoparticles as first test line 51 and first control line 53. However, in other examples, the portion of filter pad 56 between first lateral flow strip 48 and second lateral flow strip 58 may be preloaded with a different nanoparticle than the portion of filter pad 56 located between sample collection probe 22 and first lateral flow strip 48. This way the presence or concentration of multiple biomolecules of interest may be detected by assay unit 26B.

As illustrated, first lateral flow strip 48 and second lateral flow strip 58 are the same size. Additionally, the distance between sample collection probe 22 and first lateral flow strip 48 on filter pad 56 is approximately the same distance as that between first lateral flow strip 48 and second lateral flow strip 58 on filter pad 56. This means that the biological sample will flow through each lateral flow strip at approximately the same rate. For example, if it takes the biological sample about five minutes to flow from sample collection probe 22 through filter pad 56 and through first lateral flow strip 48, it should take approximately the same amount of time for the biological sample to flow from saturated first lateral flow strip 48 through filter pad 56 and through second lateral flow strip 58.

In some examples, however, it may be desirable to vary the time that it takes for the biological sample to flow through either first lateral flow strip 48 or second lateral flow strip 58. Factors that may drive this decision may include testing for the presence or concentration of a particular biomolecule at preselected time intervals.

Varying the time it takes for the biological sample to flow through first lateral flow strip 48 and second lateral flow strip 58 may be done in many ways. For example, first lateral flow strip 48 may be designed to have a greater length than second lateral flow strip 58. Alternatively, second lateral flow strip 58 may have a greater length than first lateral flow strip 48. The different lengths mean that the biological sample will flow at different rates through each lateral flow strip (e.g., the biological sample will flow through the shorter of the lateral flow strips quicker). Similarly, the width of each lateral flow strip may be varied. For example, first lateral flow strip 48 may be designed to have a greater width than second lateral flow strip 58. Alternatively, second lateral flow strip 58 may have a greater width than first lateral flow strip 48. The different widths mean that the biological sample will flow through at different rates, with the biological sample flowing through the narrower of the lateral flow strips quicker.

Another way to vary the rate at which the biological sample flows through first lateral flow strip 48 and second lateral flow strip 58 is to vary the location along filter pad 56 at which each strip is attached. For example, the distance along filter pad 56 between sample collection probe 22 and first lateral flow strip 48 may be less than the distance along filter pad 56 between first lateral flow strip 48 and second lateral flow strip 58. Alternatively, the distance along filter pad 56 between sample collection probe 22 and first lateral flow strip 48 may be greater than the distance along filter pad 56 between first lateral flow strip 48 and second lateral flow strip 58. Thus the rate of flow may be varied, in that it will take the biological sample longer to filter through the filter pad 56 to reach one of the lateral flow strips than to reach the other.

FIG. 5 shows another example assay unit 26B which includes removable blocking layer 66. Blocking layer 66 comprises a hydrophobic material. As stated above, the biological sample, particularly when it is an interstitial fluid, includes mostly water. Therefore, blocking layer 66 is able to sufficiently block the passage of the biological sample therethrough. Suitable hydrophobic materials that blocking layer 66 may be made out of include silicone elastomers, fluoropolymers, rubbers, polyvinyl chloride, polyurethane, wax, and combinations thereof. In some examples, the hydrophobic material may be about 50% (w/w) to about 100% (w/w) of blocking layer 66, or about 90% (w/w) to about 100% (w/w) of blocking layer 66, or less than about, equal to about, or greater than about 55% (w/w), 60, 65, 70, 75, 80, 85, 90, or 95% (w/w) of blocking layer 66. In some examples, the balance of blocking layer 66 includes a material that is less permeable than the hydrophobic material.

As shown, removable blocking layer 66 is disposed within filter pad 56 between first lateral flow strip 48 and second lateral flow strip 58. This will substantially prevent the biological sample from flowing from first lateral flow strip 48 to second lateral flow strip 58. Blocking layer 66 may be disposed elsewhere in assay unit 26B in order to control flow. For example, blocking layer 66 may be placed between sample collection probe 22 and first lateral flow strip 48. Additionally, blocking layer 66 may be placed between first lateral flow strip 48 and first wick pad 54. If blocking layer 66 is placed there, then the biological sample will not flow through first lateral flow strip 48. This is because first wick pad 54 may not absorb the biological sample to drive the capillary action through first lateral flow strip 48. Instead, the flow will be driven through filter pad 56 and second lateral flow strip 58. However, a second blocking layer may also be located between first lateral flow strip 48 and second lateral flow strip 58 to completely cut off the flow of the biological sample. In other examples, blocking layer 66 may be disposed between second wick pad 60 and second lateral flow strip 58 in order to prevent flow through second lateral flow strip 58.

Blocking layer 66 may be removed many different ways. For example, blocking layer 66 may be removed manually, by the wearer simply pulling blocking layer 66 out of housing 12. This may be accomplished by including a tab on blocking layer 66, which extends through a slot in housing 12. Additionally, blocking layer 66 may be removed automatically, by heating blocking layer 66 with a heater controlled by a component of printed circuit board 42. The heat will melt blocking layer 66 but will not be hot enough to damage the other components of wearable assay system 10. Removal of blocking layer 66 may occur in response to a command issued by the wearer or according to a set schedule programmed into a controller located in printed circuit board 42.

Although blocking layer 66 is described with respect to assay unit 26B, blocking layer 66 may also be incorporated into assay unit 26A. For example, blocking layer 66 may be located between any two of sample collection probe 22, sample pad 50, conjugate pad 52, first lateral flow strip 48, and first wick pad 54.

Assay unit 26B may be a stand-alone assay system that may be placed directly into contact with a biological sample. Additionally, assay unit 26B may be incorporated into wearable assay system 10. That is, assay unit 26B may be placed in actuation system 28 and selectively placed into contact with sample collection probe 22. Additionally, wearable assay system 10 may include a mixture of assay unit 26A and assay unit 26B. For example, actuation system 28 may be loaded with both assay unit 26A and assay unit 26B and adapted to position either unit in contact with sample collection probe 22.

There are many reasons to use assay unit 26B, including the following non-limiting reasons. For example, assay unit 26B allows for multiple assays to be conducted by one assay unit. Typically, lateral flow strips are individually utilized for one test. Assay unit 26B, however, allows for multiple tests to be run by the same unit. This allows a wearer to test for the presence or concentration of a preselected biomolecule at different times with the same unit. The wearer may also run multiple assays on lateral flow strips using the same assay unit without switching units. This may allow the wearer to test for multiple biomolecules of interest without having to constantly change lateral flow strips. Additionally, the vertical arrangement of first lateral flow strip 48 and second lateral flow strip 58 allows for space savings in that the lateral surface area that would have been taken up by adjacent lateral flow strips is reduced. Incorporating blocking layer 66 into assay unit 26B may also allow the wearer to selectively turn the flow of the biological sample on once the biological sample is introduced to wearable assay system 10. In other systems, there is no way to stop the flow of the biological sample once it is absorbed.

In other examples, wearable assay system 10 may include assay unit 26C, in addition to or instead of assay unit 26A and/or assay unit 26B. As schematically shown in FIG. 6, assay unit 26C is an electrode array. As depicted, assay unit 26C includes test electrode 68, reference electrode 70, and control electrode 72. Test electrode 68 is functionalized with at least one immobilized recognition molecule 74. Recognition molecule 74 is usually an antibody that is adapted to bind to predetermined biomolecule 76.

As stated previously, the predetermined biomolecule may be a protein or hormone, among other examples. Control electrode 72 is functionalized with blocking molecules 78. Blocking molecules 78 include a plurality of immobilized blocking molecules. Typically, each of blocking molecules 78 is selected to be sized to prevent predetermined biomolecule 76 from interacting with control electrode 72. Additionally blocking molecules can be sized to be substantially the same as recognition molecules 74. An example of suitable blocking molecules 78 may include VSA proteins having a thiol side chain that is bonded to control electrode 72. In some examples, control electrode 72 may be left bare.

Typically, test electrode 68 and control electrode 72 are formed from the same material. For example, test electrode 68 and control electrode 72 may be formed from gold. Additionally, test electrode 68 and control electrode 72 are substantially the same size.

Reference electrode 70 may be formed from any electrode material. Typically, reference electrode 70 is uncoated, but in some examples it may be coated. Reference electrode 70 does not have to be the same size as test electrode 68 or control electrode 72. In some examples, reference electrode 70 is approximately twice the size of either test electrode 68 or control electrode 72 individually. Typically, the size of reference electrode 70 is driven by the amount of space available where assay unit 26C is located. That is, reference electrode 70 is sized to take up the remaining space in assay unit 26C.

To deposit recognition molecule 74 on test electrode 68, a mask may be disposed over test electrode 68 leaving openings where the surface of test electrode 68 is exposed. These sites are where recognition molecules 74 are deposited. Similarly, to deposit blocking molecules 78 on control electrode 72, a mask may be disposed over control electrode 72 leaving openings where the surface of control electrode 72 is exposed. These sites are where blocking molecules 78 are deposited.

In operation of wearable assay system 10, the biological sample is supplied to assay unit 26C by sample collection probe 22. Typically, assay unit 26C will be deployed in a chamber or other space that will allow the biological sample to interact with each electrode. Each electrode acts as a capacitor. This is due primarily to the electric double layer that is formed at the interface between the electrode and the charged molecules in the biological sample. Stated briefly, the electric double layer is an electrical phenomenon that appears at the interface between a conductive electrode and an adjacent liquid electrolyte. At the interface, two layers of ions with opposing polarity form if a voltage is applied. The two layers of ions are separated by a single layer of solvent molecules that adheres to the surface of the electrode and acts like a dielectric in a conventional capacitor. The amount of electric charge stored in double-layer capacitance is linearly proportional to the applied voltage and depends primarily on the electrode surface.

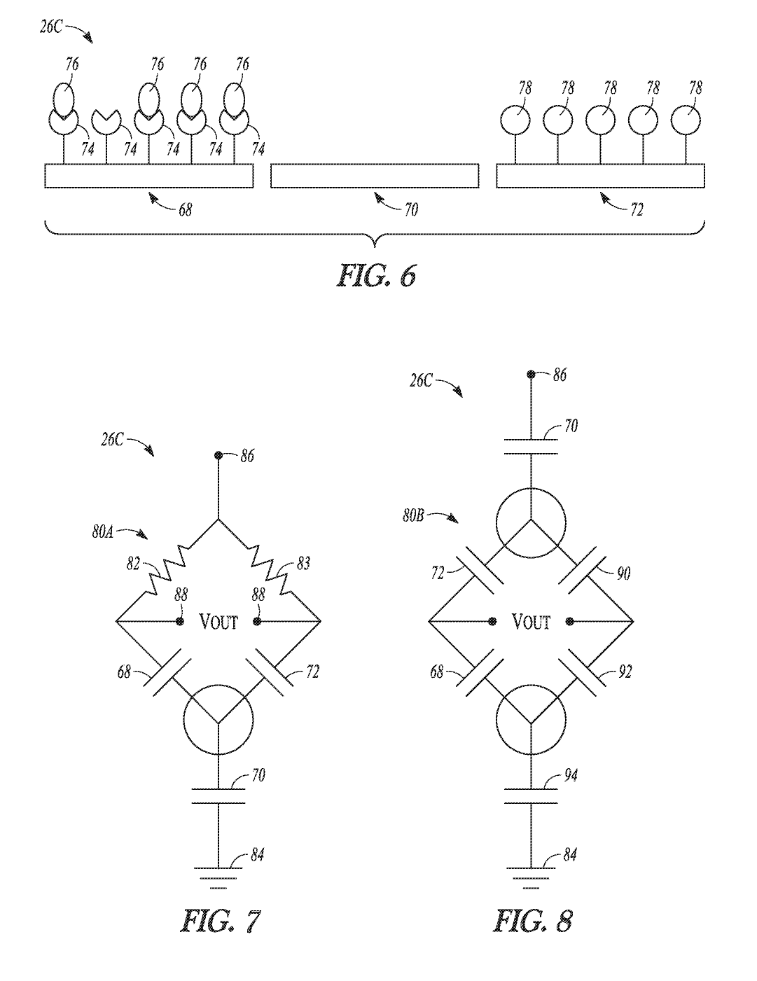

The specific capacitance of each electrode is a function of the surface charge of each electrode, the salt concentration in the biological sample, and the voltage that is applied to the electrode. The capacitance of test electrode 68 changes, however, when biomolecule 76 binds to recognition molecule 74. This is due to the displacement of ions that surround test electrode 68. To detect the changes in capacitance of test electrode 68, the electrodes are arranged in a circuit resembling a Wheatstone bridge. This circuit is shown in FIG. 7.

As shown in FIG. 7, circuit 80A includes first resistor 82, which is connected to test electrode 68. Circuit 80A further includes second resistor 83, which is connected to control electrode 72. Test electrode 68 and control electrode 72 are both connected to reference electrode 70, which in turn is connected to ground 84. Circuit 80A further includes voltage input 86 and voltage output 88. In this example, voltage input 86 is driven by an AC voltage. If a DC voltage is used instead, then circuit 80A would behave as an open circuit.

When biomolecule 76 binds to recognition molecule 74, a capacitance difference results between test electrode 68 and control electrode 72. Circuit 80A is configured such that any capacitance changes in control electrode 72 or drift in control electrode 72 caused by pH changes due to changes in the concentration of the biological sample concentration appear as a common mode of the circuit. This improves the resolution of the capacitance measured by test electrode 68 and control electrode 72. The capacitance of test electrode 68 and control electrode 72 may be detected by a component in printed circuit board 42. The capacitance difference is then measured and correlated to a concentration of biomolecule 76. Circuit 80A may allow for very precise measurements of capacitance differences up to the nanofarad range. Other circuit designs are within the scope of this disclosure. For example, FIG. 8 is a schematic diagram of circuit 80B. Circuit 80B is constructed as a full Wheatstone bridge and includes second test electrode 90, second control electrode 92, and second reference electrode 94.

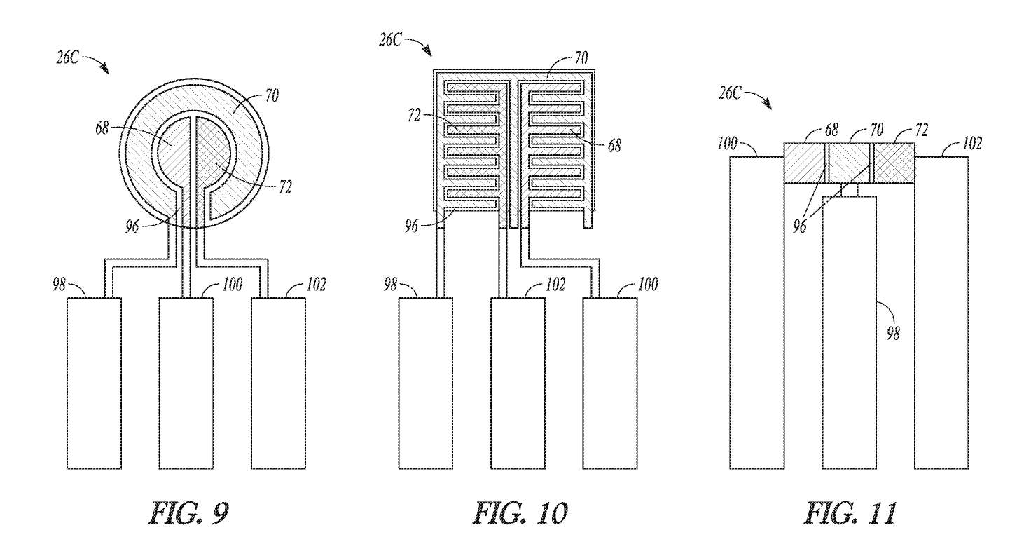

Assay unit 26C may be constructed in or configured in many different ways as either a stand-alone assay device or as part of wearable assay system 10. FIGS. 9, 10, and 11 show different examples of assay unit 26C. FIGS. 9 and 10 are schematic sectional views of assay unit 26C in which test electrode 68 and control electrode 72 are substantially encapsulated by reference electrode 70.

As shown in FIG. 9, reference electrode 70 has a circular profile defining an interior space in which test electrode 68 and control electrode 72 are positioned. Test electrode 68 and control electrode 72 are spaced apart from the interior of reference electrode 70 and from each other. The spaces form biological sample channel 96. Biological sample channel 96 provides an entrance for the biological sample to assay unit 26C. Biological sample channel 96 also provides a space for the biological sample to flow through such that the specified biomolecule 76 may bind to test electrode 68. Test electrode 68 is joined to second interconnect 100. Reference electrode 70 is joined to first interconnect 98. Control electrode 72 is joined to third interconnect 102. Each interconnect may connect to an element that may provide power to assay unit 26C or send capacitance data back to printed circuit board 42.

FIG. 10 shows an alternative arrangement of assay unit 26C in which test electrode 68, reference electrode 70, and control electrode 72 have a toothed configuration. Specifically, a projection of control electrode 72 is located between projections of reference electrode 70, and a projection of test electrode 68 is located adjacent to a projection of reference electrode 70. Biological sample channel 96 winds between each pair of projections. This configuration increases the surface areas of test electrode 68 and control electrode 72, which allows for more interaction between the biological sample and each electrode, thereby providing for increased accuracy in measuring capacitance differences.

FIG. 11 shows an additional alternative arrangement of assay unit 26C in which test electrode 68 is located adjacent to reference electrode 70 and biological sample channel 96 is disposed therebetween. Additionally, control electrode 72 is located adjacent to reference electrode 70 with biological sample channel 96 disposed therebetween. This arrangement exposes the electrodes more directly to the biological sample, as opposed to the arrangements shown in FIGS. 9 and 10, in which the biological sample must travel through biological sample channel 96. This arrangement may be desirable for applications in which less sensitivity in the measurements is required. For more sensitive applications, the arrangements shown in FIGS. 9 and 10 may be more desirable.

There are many reasons to use assay unit 26C, including the following non-limiting reasons. For example, assay unit 26C allows wearable assay system 10 to make real-time detections of the presence and concentration of biomolecule 76. Typically, in conventional immunoassay applications, such as those described herein, detection is not in real time but is delayed; for example, if lateral flow strip 48 is used, the presence of a particular biomolecule is not determined until it flows through lateral flow strip 48 to test line 51 and control line 53. In contrast, using assay unit 26C, the detection of biomolecule 76 is instantly communicated to the wearer when biomolecule 76 binds to recognition molecule 74. Depending on the concentration of biomolecule 76, the capacitance will change. Thus, the wearer may continuously monitor the concentration of biomolecule 76 over time by leaving assay unit 26C exposed to the biological sample.

Additionally, wearable assay system 10 may be adapted to include multiple assay units 26C, each of which is individually disposable, placed in communication with sample collection probe 22 by actuation system 28. For example, each assay unit 26C may be disposed in a slot of actuation system 28. Each assay unit 26C may differ from the others in terms of what recognition molecule 74 is coated on test electrode 68. Thus individual assay units 26C configured to test for the presence or concentration of different biomolecules 76 may be selectively exposed to the biological sample.

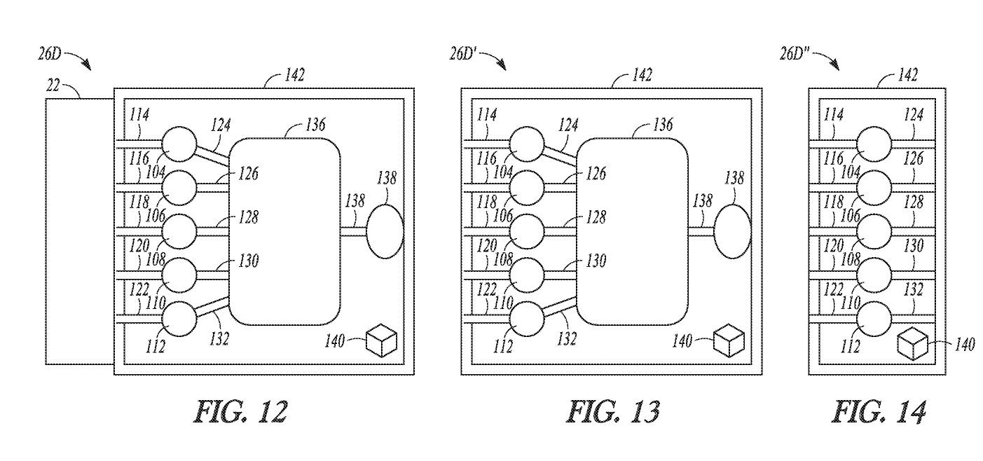

FIG. 12 shows assay unit 26D. Assay unit 26D is a modular removable cartridge that may be used in conjunction with wearable assay system 10. As shown in FIG. 12, assay unit 26D includes first well 104, second well 106, third well 108, fourth well 110, and fifth well 112. In other examples, assay unit 26D may include fewer or more wells depending on the specific application. FIG. 12 also shows sample collection probe 22, which may be directly attached to assay unit 26D. Assay unit 26D also includes first sample channel 114, second sample channel 116, third sample channel 118, fourth sample channel 120, and fifth sample channel 122. FIG. 12 also shows first reagent exit channel 124, second reagent exit channel 126, third reagent exit channel 128, fourth reagent exit channel 130, and fifth reagent exit channel 132. FIG. 12 also shows processing well 134, processing well exit channel 136, and detection well 138.

First well 104, second well 106, third well 108, fourth well 110, and fifth well 112 are connected to sample collection probe 22 by corresponding first sample channel 114, second sample channel 116, third sample channel 118, fourth sample channel 120, and fifth sample channel 122, respectively. First well 104, second well 106, third well 108, fourth well 110, and fifth well 112 are further connected to corresponding first reagent exit channel 124, second reagent exit channel 126, third reagent exit channel 128, fourth reagent exit channel 130, and fifth reagent exit channel 132, respectively. Detection well 138 is connected to processing well 134 by processing well exit channel 136. Assay unit 26D further includes reagent sensor 140, which is disposed on the interior of assay unit 26D. Assay unit 26D also includes perimeter sensor 142, which is disposed along an outer perimeter of assay unit 26D.

In operation of assay unit 26D, the biological sample enters assay unit 26D through sample collection probe 22, where it enters at least one of first well 104, second well 106, third well 108, fourth well 110, and fifth well 112. Each well may include at least one reagent for processing the biological sample. For example, each well may contain one or more antibodies configured to attach to a predetermined biomolecule. Some reagents may include organic solvents to separate organic molecules into a discrete phase. Other reagents include components of polymerase chain reaction experiments. Other reagents include molecules that are tagged with a fluorescent marker and are adapted to bind to a specific biomolecule. In some examples, the reagents are stored in each well in a lyophilized state to aid in preserving the molecule; when the liquid-phase biological sample interacts with the reagents, then the reagents are hydrated and may react with the biological sample.

After the biological sample has passed through the assay unit 26D, it passes to processing well 134. In processing well 134, the biomolecules may be mixed into one sample. Additionally, organic and inorganic phases may be separated. Also, several preprocessing steps may occur, such as concentration of the biological sample. Filters may be located in processing well 134 in order to separate solids and liquids. Heat may also be provided to processing well 134 in order to evaporate organic solvents. Finally, the biological sample may simply be stored in processing well 134.

From processing well 134, the biological sample may pass into detection well 138. Detection well 138 may be aligned with detection system 34. If detection system 34 includes a light source and photodetector, then light may be supplied to detection well 138 to produce a fluorescent emission from any preselected biomolecules that are tagged with a fluorescent molecule. Additionally, if any components of the biological sample are nucleic acids or proteins, they may be capable of emitting a fluorescent signal if light is supplied to detection well 138. Although fluorescent studies are described herein, other detection techniques may be performed at detection well 138.

Reagent sensor 140 is disposed within assay unit 26D. Reagent sensor 140 functions to monitor the conditions within assay unit 26D. For example, reagent sensor 140 may be adapted to measure the temperature, humidity, and/or pressure inside the cartridge. Reagent sensor 140 may output this data to printed circuit board 42. This data may help to monitor the stability of the reagents in the wells. That is, the conditions inside assay unit 26D may be analyzed to determine whether the reagents in the wells are exposed to conditions that may lead to decomposition or destabilization of the reagents. If these conditions are met, then a determination not to use a particular assay unit 26D may be made by wearable assay system 10 or by the wearer.

Perimeter sensor 142 is adhered to an external perimeter of assay unit 26D. Perimeter sensor 142 may sense contact between assay unit 26D and housing 12. That is, perimeter sensor 142 may determine whether assay unit 26D is properly disposed in a particular location of housing 12. Perimeter sensor 142 may affirmatively communicate this to printed circuit board 42, which may in turn allow the assay to proceed. Alternatively, if perimeter sensor 142 determines that assay unit 26D is not properly inserted in housing 12, then the particular assay may be halted. Perimeter sensor 142 may also sense whether there is any leakage of the biological sample or reagent from assay unit 26D. In the event that there is any leakage, perimeter sensor 142 may communicate this to printed circuit board 42, which in turn may halt the assay. Additionally, an alert from perimeter sensor 142 that there is any leakage may produce an audible alarm indicating to the wearer to remove wearable assay system 10 from themselves.

FIG. 13 is a schematic diagram showing an alternative configuration of assay unit 26D′. In this configuration, assay unit 26D′ is not directly coupled to sample collection probe 22. Instead, assay unit 26D′ is placed on sample collection probe 22 by actuation system 28.

FIG. 14 is a schematic diagram showing assay unit 26D″. Assay unit 26D″ includes first well 104, second well 106, third well 108, fourth well 110, and fifth well 112. In other examples, assay unit 26D″ may include fewer or more wells depending on the specific application. Assay unit 26D″ also includes first sample channel 114, second sample channel 116, third sample channel 118, fourth sample channel 120, and fifth sample channel 122. FIG. 14 also shows first reagent exit channel 124, second reagent exit channel 126, third reagent exit channel 128, fourth reagent exit channel 130, and fifth reagent exit channel 132.

Each of the sample channels may be connected to sample collection probe 22. Additionally, each of the sample channels may be further connected to one processing well or to another assay unit 26. For example, each well may be connected to one lateral flow strip 48. Alternatively, each well may be connected to a different lateral flow strip 48. In this case, each well may contain different reagents, such as different nanoparticles that are each adapted to attach to a different preselected biological molecule.

Any of assay unit 26D, assay unit 26D′, or assay unit 26D″ may be individually placed in communication with the biological sample by actuation system 28. Additionally, the assay units may be configured as individually insertable or removable cartridges in wearable assay system 10. The ability to selectively insert or remove assay unit 26D may allow for great control of and variation in the particular assay that wearable assay system 10 will perform.

Additionally, the removability of assay unit 26D may allow for the storage of a sample for future experiments. For example, the reagents in the wells could be primers and enzymes used in polymerase chain reaction (PCR) experiments. The biological sample and PCR reagents could be mixed in the wells and assay unit 26D could be removed and placed in a thermal cycling device.

Actuation system 28 has been discussed in connection with the placement of a respective assay unit in communication with sample collection probe 22. FIG. 2 shows an example of actuation system 28. As depicted, actuation system 28 includes rotating disc 144. Rotating disc 144 is attached to housing 12 and adapted to move assay unit 26 into contact with sample collection probe 22. Each assay unit 26 may be disposed within a slot on rotating disc 144. Each slot is sized to receive and secure assay unit 26.

Although actuation system 28 is shown as including rotating disc 144, other configurations are possible. For example, actuation system 28 may include a band that includes slots sized to receive individual assay units 26. Alternatively, the band could be formed from multiple assay units 26. For example, the band could be formed from a number of lateral flow strips 48. The band may be wrapped around a drum, which unwinds the band to place assay unit 26 in communication with sample collection probe 22.

Although many different types of assay units 26 are described herein, it should be understood that other suitable assay units may be used in conjunction with wearable assay system 10. Additionally, any one of the described assay units 26 may be used as a stand-alone assay device. Wearable assay system 10 may use any one of assay units 26 individually. Wearable assay system 10 may also use any number of assay units 26. Wearable assay system 10 may use any combination of assay units 26A, 26B, 26C, or 26D, 26D′, or 26D″.

Once the predetermined biomolecule is collected and processed using assay unit 26, detection system 34 may be used to detect a particular property of the predetermined biomolecule. Detection system 34 is shown in FIG. 2 and schematically in FIG. 15. As shown in FIG. 15, detection system 34 includes first optical sensor 146 and second optical sensor 148. Each optical sensor includes a light-emitting source and a light-detection source. Typically, the light-emitting source is a light-emitting diode and the light-detection source is a photodiode. The light-emitting diode may be configured to selectively deliver light at a desired wavelength. For example, the light-emitting diode may deliver light in the visible or near-visible range. In some examples, the light may be at a wavelength to effect a fluorescent emission in the biological sample. To produce the different wavelengths, a filter may be placed in front of the light-emitting diode and may be cycled through different settings to selectively filter different wavelengths of light. Alternatively, detection system 34 may include an array of light-emitting sources such as light-emitting diodes, each of which is selectively configured to deliver light at a different wavelength to the biological sample.

As shown in FIG. 15, first and second optical sensors 146 and 148 are positioned over test line 51 and control line 53, respectively. Upon receiving a command from printed circuit board 42, the light-emitting sources send light at each of test line 51 and control line 53. Any reflected or emitted light from test line 51 or control line 53 is detected by the light detection source, which is a photodetector. Data from the photodetector is processed and may be correlated with the presence of a particular biomolecule or the concentration of the biomolecule.

Pumping system 36 helps to move or pump the biological sample through wearable assay system 10. That is, pumping system 36 may help to pump the biological sample from sample collection probe 22 through wearable assay system 10. Typically, pumping system 36 creates a low-pressure vacuum-like environment within housing 12. In some examples, however, pumping system 36 generates heat within housing 12 to evaporate reagents or some constituents of the biological sample to drive movement of the biological sample. As shown in FIG. 1, pumping system 36 includes pump 38, which is adapted to pump air from the interior of housing 12 to the exterior of housing 12. By pumping air out of housing 12, pump 38 lowers the pressure inside housing 12, which may help to drive the flow of the biological sample, which usually flows via a capillary action.

Pump 38 may be driven by the same power source that powers other components of wearable assay system 10, such as a chargeable battery. However, in order to save power, other systems may be used to power pump 38.

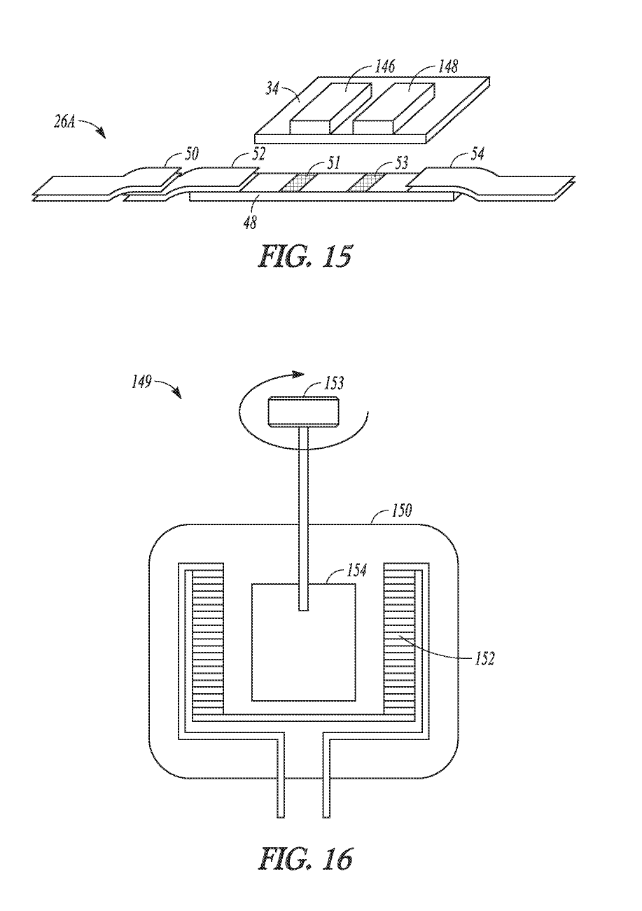

FIG. 16 shows dynamo 149 that may be used to power pump 38. A dynamo uses rotating coils of wire and magnetic fields to convert mechanical rotation into a pulsing direct electric current through Faraday's law of induction. Dynamo 149 may be directly linked to pump 38 or may be linked to a capacitor connected to pump 38. As illustrated, dynamo 149 includes body 150, which houses coiled wires 152 that are disposed along an interior surface of body 150. A void is defined within coiled wires 152, and magnet 154 is disposed therein. Magnet 154 is attached to dial 153, which extends outside of housing 12. Magnet 154 is adapted to rotate in response to movement by the wearer. Magnet 154 may also be manually rotated by the wearer spinning dial 153. As magnet 154 rotates, electrical power is generated.

In additional examples, a solar panel is located on second external surface 16 of housing 12. In still other examples, a piezoelectric element may be located in housing 12. The piezoelectric element may be adapted to be struck in response to movement by the wearer. The electrical power generated by the solar panel or piezoelectric element may be used to directly power pump 38 or stored in a capacitor. In addition to powering pump 38, the electricity generated by dynamo 149, the solar panel, or the piezoelectric element may power other systems of wearable assay system 10 such as detection system 34 or actuation system 28.

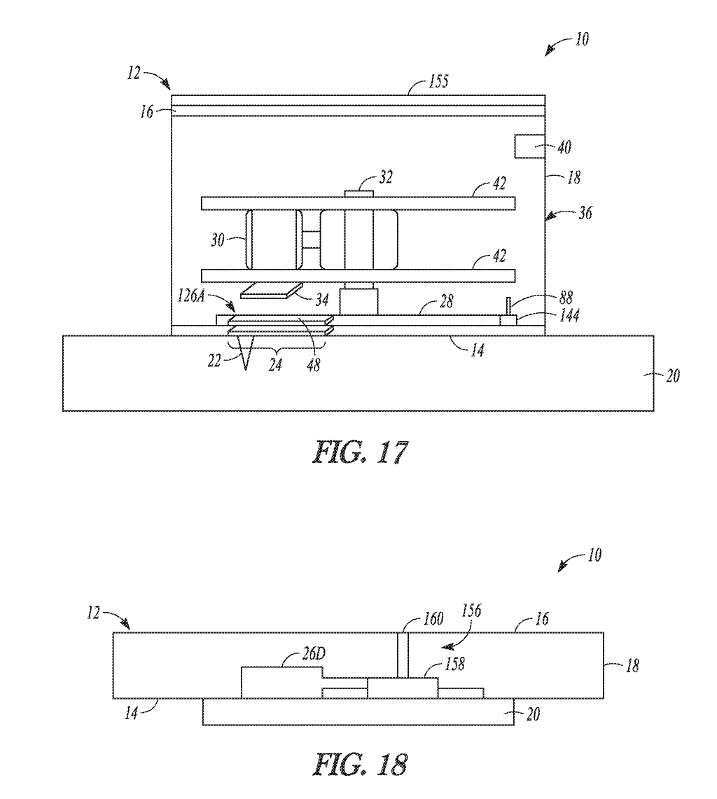

Some examples of pumping system 36 may not include pump 38 or may use other systems for pumping the biological sample. For example, FIG. 17 is a schematic view of wearable assay system 10 having a flexible membrane on an external surface of the device. As shown in FIG. 17, wearable assay system 10 includes flexible membrane 155. Flexible membrane 155 defines at least a portion of an external surface of housing 12. Wearable assay system 10 further includes vent 160. Vent 160 is a one-way vent and is adapted to expel air from housing 12. Air is expelled from housing 12 in response to the wearer depressing flexible membrane 153. Once the air is expelled, the pressure inside housing 12 is lowered. Thus the wearer may initiate pumping or help to maintain pumping without using electrical power.

Another way to pump the biological sample through wearable assay system 10 is by using transpiration, in which a fluid is evaporated under a temperature or pressure differential in wearable assay system 10. FIG. 18 shows an example of wearable assay system 10 including transpiration system 156. As shown, transpiration system 156 includes metallic layer 158 and vent 160. Metallic layer 158 is disposed on first external surface 14 of housing 12 and is adapted to contact the wearer's skin 20. Vent 160 is attached to detection system 34 and has an exit defined by second external surface 16 of housing 12.

In operation, metallic layer 158 picks up heat from the wearer. Once the heat reaches a sufficient level, volatile reagents will begin to evaporate. For example, in assay unit 26D, reagents such as alcohols or ethers may be disposed in any of the wells including detection well 138. As these reagents are evaporated, the biological sample is pumped through the wearable assay system 10.

The pumping may be accelerated by using a heater element. For example, a heater may be located adjacent to metallic layer 158 and may be selectively engaged to heat metallic layer 158. This may increase the evaporation of the volatile reagents or evaporate less volatile reagents or constituents in the biological sample.

According to various examples, a method of performing an assay using wearable assay system 10 includes contacting sample collection probe 22 with a layer of skin of a wearer. A biological sample is then collected from the wearer by sample collection probe 22. The biological sample is pumped from sample collection probe 22 to assay unit 26, where a property of the biological sample is detected.

The method may further include pumping the biological sample to a second assay unit 26. The second assay unit 26 and the first assay unit 26 may be selectively placed in fluid communication with sample collection probe 22 by actuation system 28.

### Additional Embodiments

The following exemplary embodiments are provided, the numbering of which is not to be construed as designating levels of importance:

Example 1 is a wearable assay system comprising: a housing comprising: a first external surface adapted to contact a wearer; and a void defined by a portion of the first external surface; a sample collection probe positioned near the void and attached to the first external surface and adapted to collect a biological sample from the wearer; an assay unit adapted to receive the biological sample from the sample collection probe; an actuation system adapted to position the assay unit in contact with the sample collection probe; and a detection system adapted to detect a property of the biological sample.

In Example 2, the subject matter of Example 1 optionally includes and further comprising: a second assay unit adapted to receive the biological sample from the sample collection probe.

In Example 3, the subject matter of any one or more of Examples 1-2 optionally includes wherein the housing comprises: a first attachment member; and a second attachment member.

In Example 4, the subject matter of any one or more of Examples 1-3 optionally includes wherein the first attachment member is attached to a first strap.

In Example 5, the subject matter of any one or more of Examples 1-4 optionally includes wherein the second attachment member is attached to a second strap.

In Example 6, the subject matter of any one or more of Examples 1-5 optionally includes wherein the first strap and the second strap are attached to each other.

In Example 7, the subject matter of any one or more of Examples 1-6 optionally includes wherein the first strap and the second strap are attached to each other by a buckle.

In Example 8, the subject matter of any one or more of Examples 1-7 optionally includes wherein the first strap and the second strap are attached to each other by a Velcro connection.

In Example 9, the subject matter of any one or more of Examples 1-8 optionally includes wherein at least one of the first attachment member and the second attachment member comprises: an adhesive layer disposed on the first external surface.

In Example 10, the subject matter of any one or more of Examples 1-9 optionally includes wherein the adhesive layer is adapted to adhere the wearable assay system to the wearer.

In Example 11, the subject matter of any one or more of Examples 1-10 optionally includes wherein the attachment member comprises: a flexible sheet attached to the housing and adapted to contact the wearer; and an adhesive layer disposed on a first surface of the flexible sheet.

In Example 12, the subject matter of any one or more of Examples 1-11 optionally includes wherein the adhesive layer is adapted to adhere the flexible sheet to the wearer.

In Example 13, the subject matter of any one or more of Examples 1-12 optionally includes wherein the sample collection probe is a needle.

In Example 14, the subject matter of any one or more of Examples 1-13 optionally includes wherein the needle is hollow.

In Example 15, the subject matter of any one or more of Examples 1-14 optionally includes wherein the needle has a diameter ranging from about 10 μm to about 50 μm.

In Example 16, the subject matter of any one or more of Examples 1-15 optionally includes wherein the needle has a diameter ranging from about 10 μm to about 20 pmn.

In Example 17, the subject matter of any one or more of Examples 1-16 optionally includes wherein the needle is adapted to be movable between a first position and a second position.

In Example 18, the subject matter of any one or more of Examples 1-17 optionally includes wherein a major portion of the needle is disposed outside of the housing when the needle is in the first position.

In Example 19, the subject matter of any one or more of Examples 1-18 optionally includes wherein a major portion of the needle is disposed within the housing when the needle is in the second position.

In Example 20, the subject matter of any one or more of Examples 1-19 optionally includes wherein the needle is adapted to puncture a layer of skin of the wearer.

In Example 21, the subject matter of any one or more of Examples 1-20 optionally includes wherein the biological sample is drawn through the needle by a capillary action.

In Example 22, the subject matter of any one or more of Examples 1-21 optionally includes wherein the sample collection probe is a wick pad.

In Example 23, the subject matter of any one or more of Examples 1-22 optionally includes wherein the wick pad is adapted to contact the layer of skin of the wearer.

In Example 24, the subject matter of any one or more of Examples 1-23 optionally includes wherein the wick pad is adapted to absorb sweat from the wearer.

In Example 25, the subject matter of any one or more of Examples 1-24 optionally includes wherein the wick pad is adapted to absorb a subdermal interstitial fluid that is exposed on the layer of skin of the wearer.

In Example 26, the subject matter of any one or more of Examples 1-25 optionally includes wherein the biological sample comprises a subdermal interstitial fluid.

In Example 27, the subject matter of any one or more of Examples 1-26 optionally includes wherein the subdermal interstitial fluid comprises a protein.

In Example 28, the subject matter of any one or more of Examples 1-27 optionally includes wherein the protein is about 1% (w/w) to about 5% (w/w) of the subdermal interstitial fluid.

In Example 29, the subject matter of any one or more of Examples 1-28 optionally includes wherein the subdermal interstitial fluid comprises a hormone.

In Example 30, the subject matter of any one or more of Examples 1-29 optionally includes wherein the hormone is about 1% (w/w) to about 5% (w/w) of the subdermal interstitial fluid.

In Example 31, the subject matter of any one or more of Examples 1-30 optionally includes wherein the subdermal interstitial fluid comprises a metabolite.

In Example 32, the subject matter of any one or more of Examples 1-31 optionally wherein the metabolite is about 1% (w/w) to about 5% (w/w) of the subdermal interstitial fluid.

In Example 33, the subject matter of any one or more of Examples 1-32 optionally includes % (w/w) of the subdermal interstitial fluid.

In Example 34, the subject matter of any one or more of Examples 1-33 optionally includes wherein the sweat is located on the layer of skin of the wearer.

In Example 35, the subject matter of any one or more of Examples 1-34 optionally includes wherein the sweat comprises a protein.

In Example 36, the subject matter of any one or more of Examples 1-35 optionally includes wherein the protein ranges from about 1% (w/w) to about 5% (w/w) of the sweat.

In Example 37, the subject matter of any one or more of Examples 1-36 optionally includes wherein the protein ranges from about 1% (w/w) to about 2% (w/w) of the sweat.

In Example 38, the subject matter of any one or more of Examples 1-37 optionally includes wherein the sweat comprises a hormone.

In Example 39, the subject matter of any one or more of Examples 1-38 optionally includes wherein the hormone ranges from about 1% (w/w) to about 5% (w/w) of the sweat.

In Example 40, the subject matter of any one or more of Examples 1-39 optionally includes wherein the assay unit is adapted to test for different biological properties.

In Example 41, the subject matter of any one or more of Examples 1-40 optionally includes wherein the assay unit comprises a lateral flow strip.

In Example 42, the subject matter of any one or more of Examples 1-41 optionally includes wherein the assay unit comprises an array of lateral flow strips.

In Example 43, the subject matter of any one or more of Examples 1-42 optionally includes and further comprising: an absorbent sample pad that receives the biological sample from the needle; and an absorbent conjugate pad that receives the biological sample from the sample pad, wherein the conjugate pad comprises a plurality of nanoparticles configured to bind to a preselected component of the biological sample.

In Example 44, the subject matter of any one or more of Examples 1-43 optionally includes wherein a first end of the lateral flow strip is in contact with the conjugate pad and a second end of the lateral flow strip is in contact with a second wick pad.

In Example 45, the subject matter of any one or more of Examples 1-44 optionally includes wherein the lateral flow strip comprises: a test line adapted to attach to nanoparticles bound to the preselected component of the biological sample; and a control line adapted to attach to nanoparticles that are free of the biological sample.

In Example 46, the subject matter of any one or more of Examples 1-45 optionally includes wherein the nanoparticles comprise antibodies.

In Example 47, the subject matter of any one or more of Examples 1-46 optionally includes wherein the test line comprises an activated surface.

In Example 48, the subject matter of any one or more of Examples 1-47 optionally includes wherein the activated surface comprises an antibody.

In Example 49, the subject matter of any one or more of Examples 1-48 optionally includes wherein the antibody is adapted to bind to a preselected component of the biological sample.

In Example 50, the subject matter of any one or more of Examples 1-49 optionally includes wherein the array of lateral flow strips comprises: a filter pad adapted to contact the sample collection probe; a first lateral flow strip attached to a first location of the filter pad; and a second lateral flow strip attached to a second location of the filter pad.

In Example 51, the subject matter of any one or more of Examples 1-50 optionally includes wherein the first lateral flow strip is joined to the filter pad at a first end and to a first wicking pad at a second end.

In Example 52, the subject matter of any one or more of Examples 1-51 optionally includes wherein the second lateral flow strip is joined to the filter pad at a first end and to a second wicking pad at a second end.

In Example 53, the subject matter of any one or more of Examples 1-52 optionally includes wherein the first lateral flow strip and the second lateral flow strip are the same size.

In Example 54, the subject matter of any one or more of Examples 1-53 optionally includes wherein the first lateral flow strip has a greater length than the second lateral flow strip.

In Example 55, the subject matter of any one or more of Examples 1-54 optionally includes wherein the second lateral flow strip has a greater length than the first lateral flow strip.

In Example 56, the subject matter of any one or more of Examples 1-55 optionally includes wherein the first lateral flow strip has a greater width than the second lateral flow strip.

In Example 57, the subject matter of any one or more of Examples 1-56 optionally includes wherein the second lateral flow strip has a greater width than the first lateral flow strip.

In Example 58, the subject matter of any one or more of Examples 1-57 optionally includes and further comprising: a removable blocking pad disposed within the filter pad.

In Example 59, the subject matter of any one or more of Examples 1-58 optionally includes wherein the blocking pad is disposed between the first lateral flow strip and the second lateral flow strip.

In Example 60, the subject matter of any one or more of Examples 1-59 optionally includes wherein the blocking pad is disposed between the sample collection probe and the first lateral flow strip.

In Example 61, the subject matter of any one or more of Examples 1-60 optionally includes wherein the blocking pad comprises a hydrophobic material.

In Example 62, the subject matter of any one or more of Examples 1-61 optionally includes wherein the hydrophobic material is about 50% (w/w) to about 100% (w/w) of the blocking pad.

In Example 63, the subject matter of any one or more of Examples 1-62 optionally includes wherein the hydrophobic material is about 90% (w/w) to about 100% (w/w) of the blocking pad.

In Example 64, the subject matter of any one or more of Examples 1-63 optionally includes wherein the blocking pad comprises a material that is less permeable than the material comprising the filter pad.

In Example 65, the subject matter of any one or more of Examples 1-64 optionally includes wherein the assay unit is an electrode array.

In Example 66, the subject matter of any one or more of Examples 1-65 optionally includes wherein the electrode array comprises: a test electrode; a reference electrode; and a control electrode.

In Example 67, the subject matter of any one or more of Examples 1-66 optionally includes wherein the test electrode is functionalized with a recognition molecule.

In Example 68, the subject matter of any one or more of Examples 1-67 optionally includes wherein the test electrode is functionalized with a second recognition molecule.

In Example 69, the subject matter of any one or more of Examples 1-68 optionally includes wherein the recognition molecule is an antibody adapted to bind to a preselected component of the biological sample.

In Example 70, the subject matter of any one or more of Examples 1-69 optionally includes wherein the second recognition molecule is a second antibody adapted to bind to a second preselected molecule of the biological sample.

In Example 71, the subject matter of any one or more of Examples 1-70 optionally includes wherein the reference electrode is free of any recognition molecules.

In Example 72, the subject matter of any one or more of Examples 1-71 optionally includes wherein the control electrode is functionalized with a blocking layer.

In Example 73, the subject matter of any one or more of Examples 1-72 optionally includes wherein the blocking layer is free of any bound preselected molecules.

In Example 74, the subject matter of any one or more of Examples 1-73 optionally includes wherein the blocking layer comprises a VSA protein.

In Example 75, the subject matter of any one or more of Examples 1-74 optionally includes wherein the test electrode and the control electrode are substantially the same size.

In Example 76, the subject matter of any one or more of Examples 1-75 optionally includes wherein the reference electrode is larger than at least one of the test electrode and the control electrode.

In Example 77, the subject matter of any one or more of Examples 1-76 optionally includes wherein the test electrode and the reference electrode are formed from the same material.

In Example 78, the subject matter of any one or more of Examples 1-77 optionally includes and further comprising: a first resistor connected to the test electrode.

In Example 79, the subject matter of any one or more of Examples 1-78 optionally includes and further comprising: a second resistor connected to the control electrode.

In Example 80, the subject matter of any one or more of Examples 1-79 optionally includes and further comprising: a voltage input connected to the first resistor and the second resistor, wherein the voltage input is adapted to deliver an alternating current voltage to the first resistor and the second resistor.

In Example 81, the subject matter of any one or more of Examples 1-80 optionally includes and further comprising: a voltage output.

In Example 82, the subject matter of any one or more of Examples 1-81 optionally includes and further comprising: an analog to digital converter connected to the voltage output.

In Example 83, the subject matter of any one or more of Examples 1-82 optionally includes wherein the reference electrode at least partially encases the test electrode and the control electrode.

In Example 84, the subject matter of any one or more of Examples 1-83 optionally includes wherein a micro channel is formed between the reference electrode and the test electrode.

In Example 85, the subject matter of any one or more of Examples 1-84 optionally includes wherein a micro channel is formed between the reference electrode and the control electrode.

In Example 86, the subject matter of any one or more of Examples 1-85 optionally includes wherein a micro channel is formed between the test electrode and the control electrode.

In Example 87, the subject matter of any one or more of Examples 1-86 optionally includes wherein the reference electrode is connected to an electrical ground.

In Example 88, the subject matter of any one or more of Examples 1-87 optionally includes and further comprising: a second test electrode; a second reference electrode; and a second control electrode.

In Example 89, the subject matter of any one or more of Examples 1-88 optionally includes wherein the test electrode is formed from gold.

In Example 90, the subject matter of any one or more of Examples 1-89 optionally includes wherein the second test electrode is functionalized with a recognition molecule.

In Example 91, the subject matter of any one or more of Examples 1-90 optionally includes wherein the second reference electrode is free of any recognition molecules.

In Example 92, the subject matter of any one or more of Examples 1-91 optionally includes wherein the second control electrode is functionalized with a second blocking layer.

In Example 93, the subject matter of any one or more of Examples 1-92 optionally includes wherein the second blocking layer does not bind to the preselected component.

In Example 94, the subject matter of any one or more of Examples 1-93 optionally includes wherein the blocking layer comprises a VSA protein.

In Example 95, the subject matter of any one or more of Examples 1-94 optionally includes wherein the second test electrode and the second control electrode are substantially the same size as the first test electrode and the first test electrode.

In Example 96, the subject matter of any one or more of Examples 1-95 optionally includes wherein the second reference electrode is larger than at least one of the second test electrode and the second control electrode.

In Example 97, the subject matter of any one or more of Examples 1-96 optionally includes wherein the assay unit comprises a reagent cartridge.

In Example 98, the subject matter of any one or more of Examples 1-97 optionally includes wherein the reagent cartridge comprises: a sample channel connected to the sample collection probe; a reagent well connected to the sample channel; and a reagent well exit channel connected to the reagent well.

In Example 99, the subject matter of any one or more of Examples 1-98 optionally includes wherein the reagent well comprises a reagent for processing a predetermined biomolecule.

In Example 100, the subject matter of any one or more of Examples 1-99 optionally includes wherein the reagent is an antibody.

In Example 101, the subject matter of any one or more of Examples 1-100 optionally includes wherein the cartridge further comprises: a second sample channel connected to the sample collection probe; a second reagent well connected to the second sample channel; and a second reagent well exit channel connected to the second reagent well.

In Example 102, the subject matter of any one or more of Examples 1-101 optionally includes wherein the cartridge further comprises: a sensor adapted to measure at least one of a temperature inside the cartridge, a humidity inside the cartridge, and a pressure inside the cartridge.

In Example 103, the subject matter of any one or more of Examples 1-102 optionally includes wherein the cartridge further comprises: a sensor adhered to an external perimeter of the cartridge adapted to sense at least one of contact between the cartridge and the housing, and leakage of the biological sample or the reagent from the cartridge.

In Example 104, the subject matter of any one or more of Examples 1-103 optionally includes wherein the cartridge further comprises: a processing well connected to the reagent well by the reagent well exit channel; a detection well aligned with the detection system; and a processing well exit channel connecting the processing well and the detection well.

In Example 105, the subject matter of any one or more of Examples 1-104 optionally includes wherein the reagent well and a second reagent well contain different reagents.

In Example 106, the subject matter of any one or more of Examples 1-105 optionally includes wherein the reagent is stored in at least one of the first and second the reagent wells in a lyophilized form.

In Example 107, the subject matter of any one or more of Examples 1-106 optionally includes wherein the actuation system comprises: a rotating disc attached to the housing and adapted to move the assay unit into contact with the sample collection probe.

In Example 108, the subject matter of any one or more of Examples 1-107 optionally includes wherein the rotating disc comprises: a slot adapted to receive the assay unit.

In Example 109, the subject matter of any one or more of Examples 1-108 optionally includes wherein the actuation system comprises: a continuous band of assay units.

In Example 110, the subject matter of any one or more of Examples 1-109 optionally includes wherein the band is wrapped around a roller attached to the housing.

In Example 111, the subject matter of any one or more of Examples 1-110 optionally includes wherein the detection system comprises: a light emission source; and a photodiode.

In Example 112, the subject matter of any one or more of Examples 1-111 optionally includes wherein the detection system further comprises: a second light emission source; and a second photodiode.

In Example 113, the subject matter of any one or more of Examples 1-112 optionally includes wherein the property of the biological sample is a concentration of a component of the biological sample.

In Example 114, the subject matter of any one or more of Examples 1-113 optionally includes and further comprising: a printed circuit board adapted to command at least one of the actuation system and the detection system.

In Example 115, the subject matter of any one or more of Examples 1-114 optionally includes and further comprising: a motor adapted to drive the actuation system, wherein the motor is further connected to the printed circuit board.

In Example 116, the subject matter of any one or more of Examples 1-115 optionally includes and further comprising: a pumping system.

In Example 117, the subject matter of any one or more of Examples 1-116 optionally includes wherein the pumping system comprises a micropump adapted to pump air from an interior of the housing to an exterior of the housing.

In Example 118, the subject matter of any one or more of Examples 1-117 optionally includes wherein the micropump comprises: a dynamo adapted to generate electrical power and deliver the electrical power to the micropump.

In Example 119, the subject matter of any one or more of Examples 1-118 optionally includes wherein the micropump further comprises: a capacitor connected to the dynamo and the micropump, wherein the capacitor is adapted to store the electrical power generated by the dynamo.

In Example 120, the subject matter of any one or more of Examples 1-119 optionally includes wherein the dynamo comprises: a body; a coil of wires disposed along an interior surface of the body and defining a second void; and a magnet disposed within the second void.

In Example 121, the subject matter of any one or more of Examples 1-120 optionally includes and further comprising: a dial attached to the magnet and extending outside of the body.

In Example 122, the subject matter of any one or more of Examples 1-121 optionally includes wherein the magnet is adapted to rotate in response to rotation of the dial.

In Example 123, the subject matter of any one or more of Examples 1-122 optionally includes wherein the micropump further comprises: a solar panel located on a second external surface of the housing; and a capacitor located within the housing and connected to the solar panel and the micropump.

In Example 124, the subject matter of any one or more of Examples 1-123 optionally includes wherein the micropump further comprises: a piezoelectric element connected to the micropump; and a striking element adapted to contact the piezoelectric element.

In Example 125, the subject matter of any one or more of Examples 1-124 optionally includes wherein the pumping system comprises: a flexible membrane defining at least a portion of an external surface of the housing; and a one-way vent adapted to expel air from the housing.

In Example 126, the subject matter of any one or more of Examples 1-125 optionally includes wherein the one-way vent is adapted to expel air from the housing in response to the flexible membrane being depressed.

In Example 127, the subject matter of any one or more of Examples 1-126 optionally includes and further comprising: a metallic layer attached to the first external surface of the housing; and a vent defined by a second external surface of the housing.

In Example 128, the subject matter of any one or more of Examples 1-127 optionally includes wherein the metallic layer is adapted to contact the wearer and absorb heat from the wearer.

In Example 129, the subject matter of any one or more of Examples 1-128 optionally includes wherein the heat is transferred to the assay unit.

In Example 130, the subject matter of any one or more of Examples 1-129 optionally includes and further comprising: a microheater in contact with the metallic layer.

Example 131 is a wearable assay system comprising: a housing comprising: a first external surface adapted to contact a wearer; and a void defined by a portion of the first external surface; a sample collection probe positioned near the void and attached to the first external surface and adapted to collect a biological sample from the wearer; an assay unit comprising: an array of lateral flow strips adapted to receive the biological sample from the sample collection probe; and an actuation system adapted to position the assay unit in contact with the sample collection probe.

In Example 132, the subject matter of Example 131 optionally includes wherein the array of lateral flow strips comprises: a filter pad adapted to contact the sample collection probe; a first lateral flow strip attached to a first location of the filter pad; and a second lateral flow strip attached to a second location of the filter pad.

In Example 133, the subject matter of any one or more of Examples 131-132 optionally includes wherein the first lateral flow strip is joined to the filter pad at a first end and to a first wicking pad at a second end.

In Example 134, the subject matter of any one or more of Examples 131-133 optionally includes wherein the second lateral flow strip is joined to the filter pad at a first end and to a second wicking pad at a second end.

In Example 135, the subject matter of any one or more of Examples 131-134 optionally includes wherein the first lateral flow strip and the second lateral flow strip are the same size.

In Example 136, the subject matter of any one or more of Examples 131-135 optionally includes wherein the first lateral flow strip has a greater length than the second lateral flow strip.

In Example 137, the subject matter of any one or more of Examples 131-136 optionally includes wherein the second lateral flow strip has a greater length than the first lateral flow strip.

In Example 138, the subject matter of any one or more of Examples 131-137 optionally includes wherein the first lateral flow strip has a greater width than the second lateral flow strip.

In Example 139, the subject matter of any one or more of Examples 131-138 optionally includes wherein the second lateral flow strip has a greater width than the first lateral flow strip.

Example 140 is a wearable assay system comprising: a housing comprising: a first external surface adapted to contact a wearer; and a void defined by a portion of the first external surface; a sample collection probe positioned near the void and attached to the first external surface and adapted to collect a biological sample from the wearer; and an assay unit comprising: an array of lateral flow strips adapted to receive the biological sample from the sample collection probe, wherein the array of lateral flow strips comprises: a filter pad adapted to contact the sample collection probe; a first lateral flow strip attached to a first location of the filter pad; and a second lateral flow strip attached to a second location of the filter pad.

In Example 141, the subject matter of Example 140 optionally includes wherein a blocking pad is disposed between the first lateral flow strip and the second lateral flow strip.

In Example 142, the subject matter of any one or more of Examples 140-141 optionally includes wherein the blocking pad is disposed between the sample collection probe and the first lateral flow strip.

In Example 143, the subject matter of any one or more of Examples 140-142 optionally includes wherein the blocking pad comprises a hydrophobic material.

In Example 144, the subject matter of any one or more of Examples 140-143 optionally includes wherein the hydrophobic material is about 50% (wvw) to about 100% (w/w) of the blocking pad.

In Example 145, the subject matter of any one or more of Examples 140-144 optionally includes wherein the hydrophobic material is about 90% (w/w) to about 100% (w/w) of the blocking pad.

In Example 146, the subject matter of any one or more of Examples 140-145 optionally includes wherein the blocking pad comprises a material that is less permeable than the material comprising the filter pad.

Example 147 is a wearable assay system comprising: a housing comprising: a first external surface adapted to contact a wearer; and a void defined by a portion of the first external surface; a sample collection probe positioned near the void and attached to the first external surface and adapted to collect a biological sample from the wearer; and an assay unit adapted to receive the biological sample from the sample collection probe, the assay unit comprising: an electrode array comprising: a test electrode; a reference electrode; and a control electrode.

In Example 148, the subject matter of Example 147 optionally includes wherein the test electrode is functionalized with a recognition molecule.

In Example 149, the subject matter of any one or more of Examples 147-148 optionally includes wherein the test electrode is functionalized with a second recognition molecule.

In Example 150, the subject matter of any one or more of Examples 147-149 optionally includes wherein the recognition molecule is an antibody adapted to bind to a preselected molecule of the biological sample.

In Example 151, the subject matter of any one or more of Examples 147-150 optionally includes wherein the second recognition molecule is a second antibody adapted to bind to a second preselected molecule of the biological sample.

In Example 152, the subject matter of any one or more of Examples 147-151 optionally includes wherein the reference electrode is free of any recognition molecules.

In Example 153, the subject matter of any one or more of Examples 147-152 optionally includes wherein the control electrode is functionalized with a blocking layer.

In Example 154, the subject matter of any one or more of Examples 147-153 optionally includes wherein the blocking layer is free of any bound preselected molecules.

In Example 155, the subject matter of any one or more of Examples 147-154 optionally includes wherein the blocking layer comprises a VSA protein.

In Example 156, the subject matter of any one or more of Examples 147-155 optionally includes wherein the test electrode and the control electrode are substantially the same size.

In Example 157, the subject matter of any one or more of Examples 147-156 optionally includes wherein the reference electrode is larger than at least one of the test electrode and the control electrode.

In Example 158, the subject matter of any one or more of Examples 147-157 optionally includes wherein the test electrode and the reference electrode are formed from the same material.

In Example 159, the subject matter of any one or more of Examples 147-158 optionally includes and further comprising: a second test electrode; a second reference electrode; and a second control electrode.

In Example 160, the subject matter of any one or more of Examples 147-159 optionally includes wherein the test electrode is formed from gold.

In Example 161, the subject matter of any one or more of Examples 147-160 optionally includes wherein the second test electrode is functionalized with a second recognition molecule.

In Example 162, the subject matter of any one or more of Examples 147-161 optionally includes wherein the second reference electrode is free of any recognition molecules.

In Example 163, the subject matter of any one or more of Examples 147-162 optionally includes wherein the second control electrode is functionalized with a blocking layer.

In Example 164, the subject matter of any one or more of Examples 147-163 optionally includes wherein the blocking layer does not bind to the first and second preselected molecules.

In Example 165, the subject matter of any one or more of Examples 147-164 optionally includes wherein the blocking layer comprises a VSA protein.

In Example 166, the subject matter of any one or more of Examples 147-165 optionally includes wherein the second test electrode and the second control electrode are substantially the same size as the first test electrode and the first control electrode.

In Example 167, the subject matter of any one or more of Examples 147-166 optionally includes wherein the second reference electrode is larger than at least one of the second test electrode and the second control electrode.

Example 168 is a wearable assay system comprising: a housing comprising: a first external surface adapted to contact a wearer; and a void defined by a portion of the first external surface; a sample collection probe positioned near the void and attached to the first external surface and adapted to collect a biological sample from the wearer; and an assay unit adapted to receive the biological sample from the sample collection probe, the assay unit comprising: a reagent cartridge comprising: a sample channel connected to the sample collection probe; a reagent well connected to the sample channel; and a reagent well exit channel connected to the reagent well.

In Example 169, the subject matter of Example 168 optionally includes wherein the reagent well comprises a reagent for processing a predetermined biomolecule.

In Example 170, the subject matter of any one or more of Examples 168-169 optionally includes wherein the reagent is an antibody.

In Example 171, the subject matter of any one or more of Examples 168-170 optionally includes wherein the cartridge further comprises: a second sample channel connected to the sample collection probe; a second reagent well connected to the second sample channel; and a second reagent well exit channel connected to the second reagent well.

In Example 172, the subject matter of any one or more of Examples 168-171 optionally includes wherein the cartridge further comprises: a sensor adapted to measure at least one of a temperature inside the cartridge, a humidity inside the cartridge, and a pressure inside the cartridge.

In Example 173, the subject matter of any one or more of Examples 168-172 optionally includes wherein the cartridge further comprises: a sensor adhered to an external perimeter of the cartridge and adapted to sense at least one of contact between the cartridge and the housing, and leakage of the biological sample or reagent from the cartridge.

In Example 174, the subject matter of any one or more of Examples 168-173 optionally includes wherein the cartridge further comprises: a processing well connected to the reagent well by the reagent well exit channel; a detection well aligned with a detection system; and a processing well exit channel connecting the processing well and the detection well.

In Example 175, the subject matter of any one or more of Examples 168-174 optionally includes wherein the reagent well and the second reagent well contain different reagents.

In Example 176, the subject matter of any one or more of Examples 168-175 optionally includes wherein the reagent is stored in at least one of the reagent well and the second reagent well in a lyophilized form.

In Example 177, the subject matter of any one or more of Examples 168-176 optionally includes and further comprising: a metallic layer attached to the first external surface of the housing; and a vent defined by a second external surface of the housing.

In Example 178, the subject matter of any one or more of Examples 168-177 optionally includes wherein the metallic layer is adapted to contact the wearer and absorb heat from the wearer.

In Example 179, the subject matter of any one or more of Examples 168-178 optionally includes wherein the heat is transferred to the assay unit.

In Example 180, the subject matter of any one or more of Examples 168-179 optionally includes and further comprising: a microheater in contact with the metallic layer.

Example 181 is a wearable assay system comprising: a housing comprising: a first external surface adapted to contact a wearer; and a void defined by a portion of the first external surface; a sample collection probe positioned near the void and attached to the first external surface and adapted to collect a biological sample from the wearer; an assay unit adapted to receive the biological sample from the sample collection probe; an actuation system adapted to position the assay unit in contact with the sample collection probe; and a pumping system adapted to pump the biological sample through the wearable assay system.

In Example 182, the subject matter of Example 181 optionally includes wherein the pumping system comprises: a micropump adapted to pump air from an interior of the housing to an exterior of the housing.

In Example 183, the subject matter of any one or more of Examples 181-182 optionally includes wherein the micropump comprises: a dynamo adapted to generate electrical power and deliver the electrical power to the micropump.

In Example 184, the subject matter of any one or more of Examples 181-183 optionally includes wherein the micropump further comprises: a capacitor connected to the dynamo and the micropump, wherein the capacitor is adapted to store the electrical power generated by the dynamo.

In Example 185, the subject matter of any one or more of Examples 181-184 optionally includes wherein the dynamo comprises: a body; a coil of wires disposed along an interior surface of the body and defining a second void; and a magnet disposed within the second void.

In Example 186, the subject matter of any one or more of Examples 181-185 optionally includes and further comprising: a dial attached to the magnet and extending outside of the body.

In Example 187, the subject matter of any one or more of Examples 181-186 optionally includes wherein the magnet is adapted to rotate in response to rotation of the dial.

In Example 188, the subject matter of any one or more of Examples 181-187 optionally includes wherein the pumping system comprises: a flexible membrane defining at least a portion of an external surface of the housing; and a one-way vent adapted to expel air from the housing.

Example 189 is a method of performing an assay on a wearable device comprising: contacting a sample collection probe with a layer of skin of a wearer; collecting a biological sample from the wearer with the sample collection probe; pumping the biological sample from the sample collection probe to an assay unit; and detecting a property of the biological sample.

In Example 190, the subject matter of Example 189 optionally includes and further comprising: pumping the biological sample to a second assay unit.

In Example 191, the subject matter of any one or more of Examples 189-190 optionally includes wherein the assay unit and the second assay unit are selectively placed in fluid communication with the sample collection probe by an actuation system.

In Example 192, the subject matter of any one or more of Examples 189-191 optionally includes wherein the actuation system comprises: a rotating disc attached to a housing and adapted to move the assay unit into contact with the sample collection probe.

In Example 193, the subject matter of any one or more of Examples 189-192 optionally includes wherein the rotating disc comprises: a slot adapted to receive the assay unit.

In Example 194, the subject matter of any one or more of Examples 189-193 optionally includes wherein the actuation system comprises: a continuous band formed from the assay unit.

In Example 195, the subject matter of any one or more of Examples 189-194 optionally includes wherein the band is wrapped around a roller.

In Example 196, the subject matter of any one or more of Examples 189-195 optionally includes wherein the sample collection probe is a needle.

In Example 197, the subject matter of any one or more of Examples 189-196 optionally includes wherein the needle is adapted to be movable between a first position and a second position.

In Example 198, the subject matter of any one or more of Examples 189-197 optionally includes wherein a major portion of the needle is disposed outside of the housing of the device when the needle is in the first position.

In Example 199, the subject matter of any one or more of Examples 189-198 optionally includes wherein a major portion of the needle is disposed within the housing when the needle is in the second position.

In Example 200, the subject matter of any one or more of Examples 189-199 optionally includes wherein the needle is adapted to puncture the layver of skin of the wearer.

In Example 201, the subject matter of any one or more of Examples 189-200 optionally includes wherein the biological sample is pumped from the needle to the assay unit by capillary action.

In Example 202, the subject matter of any one or more of Examples 189-201 optionally includes wherein the sample collection probe is a wick pad.

In Example 203, the subject matter of any one or more of Examples 189-202 optionally includes wherein the wick pad is adapted to contact the layer of skin of the wearer.

In Example 204, the subject matter of any one or more of Examples 189-203 optionally includes wherein the wick pad is adapted to absorb sweat from the wearer.

In Example 205, the subject matter of any one or more of Examples 189-204 optionally includes wherein the biological sample is pumped from the wick pad to the assay unit by capillary action.

In Example 206, the subject matter of any one or more of Examples 189-205 optionally includes wherein the wick pad is adapted to absorb a subdermal interstitial fluid that is exposed on the layer of skin of the wearer.

In Example 207, the subject matter of any one or more of Examples 189-206 optionally includes wherein the biological sample comprises the subdermal interstitial fluid.

In Example 208, the subject matter of any one or more of Examples 189-207 optionally includes wherein the assay unit comprises a lateral flow strip.

In Example 209, the subject matter of any one or more of Examples 189-208 optionally includes wherein the assay unit comprises an array of lateral flow strips.

In Example 210, the subject matter of any one or more of Examples 189-209 optionally includes wherein the array of lateral flow strips comprises: a filter pad adapted to contact the sample collection probe; a first lateral flow strip attached to a first location of the filter pad; and a second lateral flow strip attached to a second location of the filter pad.

In Example 211, the subject matter of any one or more of Examples 189-210 optionally includes wherein the first lateral flow strip is joined to the filter pad at a first end and to a first wicking pad at a second end.

In Example 212, the subject matter of any one or more of Examples 189-211 optionally includes wherein the second lateral flow strip is joined to the filter pad at a first end and to a second wicking pad at a second end.

In Example 213, the subject matter of any one or more of Examples 189-212 optionally includes and further comprising: pumping the biological sample through the first lateral flow strip; saturating the first lateral flow strip; and pumping the biological sample through the second lateral flow strip.

In Example 214, the subject matter of any one or more of Examples 189-213 optionally includes and further comprising: varying a rate at which the biological sample is pumped through the first and second lateral flow strip.

In Example 215, the subject matter of any one or more of Examples 189-214 optionally includes wherein the first lateral flow strip and the second lateral flow strip are the same size.

In Example 216, the subject matter of any one or more of Examples 189-215 optionally includes wherein a rate at which the biological sample is pumped through the first lateral flow strip and a rate at which the biological sample is pumped through the second lateral flow strip are substantially equivalent.

In Example 217, the subject matter of any one or more of Examples 189-216 optionally includes wherein the first lateral flow strip has a greater length than the second lateral flow strip.

In Example 218, the subject matter of any one or more of Examples 189-217 optionally includes wherein a rate at which the biological sample is pumped through the first lateral flow strip is greater than a rate at which the biological sample is pumped through the second lateral flow strip.

In Example 219, the subject matter of any one or more of Examples 189-218 optionally includes wherein the second lateral flow strip has a greater length than the first lateral flow strip.

In Example 220, the subject matter of any one or more of Examples 189-219 optionally includes wherein a rate at which the biological sample is pumped through the second lateral flow strip is greater than a rate at which the biological sample is pumped through the first lateral flow strip.

In Example 221, the subject matter of any one or more of Examples 189-220 optionally includes wherein the first lateral flow strip has a greater width than the second lateral flow strip.

In Example 222, the subject matter of any one or more of Examples 189-221 optionally includes wherein a rate at which the biological sample is pumped through the second lateral flow strip is less than a rate at which the biological sample is pumped through the first lateral flow strip.

In Example 223, the subject matter of any one or more of Examples 189-222 optionally includes wherein the second lateral flow strip has a greater width than the first lateral flow strip.

In Example 224, the subject matter of any one or more of Examples 189-223 optionally includes wherein a rate at which the biological sample is pumped through the first lateral flow strip is less than a rate at which the biological sample is pumped through the second lateral flow strip.

In Example 225, the subject matter of any one or more of Examples 189-224 optionally includes and further comprising: disposing a removable blocking pad within the filter pad.

In Example 226, the subject matter of any one or more of Examples 189-225 optionally includes wherein the blocking pad is disposed between the first lateral flow strip and the second lateral flow strip.

In Example 227, the subject matter of any one or more of Examples 189-226 optionally includes wherein the blocking pad is disposed between the sample collection probe and the first lateral flow strip.

In Example 228, the subject matter of any one or more of Examples 189-227 optionally includes wherein the blocking pad comprises a hydrophobic material.

In Example 229, the subject matter of any one or more of Examples 189-228 optionally includes wherein the blocking pad substantially prevents the biological sample from being pumped beyond the blocking pad.

In Example 230, the subject matter of any one or more of Examples 189-229 optionally includes and further comprising: removing the blocking pad from the filter pad.

In Example 231, the subject matter of any one or more of Examples 189-230 optionally includes wherein removing the blocking pad is manually accomplished by the wearer.

In Example 232, the subject matter of any one or more of Examples 189-231 optionally includes wherein removing the blocking pad is automatically accomplished by the device in response to a command issued by a printed circuit board.

In Example 233, the subject matter of any one or more of Examples 189-232 optionally includes wherein removing the blocking pad is accomplished by heating the blocking pad.

In Example 234, the subject matter of any one or more of Examples 189-233 optionally includes wherein detecting the property of the biological sample comprises: measuring a capacitance of a test electrode adapted to bind to a predetermined biological molecule; measuring a capacitance of a reference electrode adapted to be free of the predetermined biological molecule; and determining a difference in the measured capacitance between the test electrode and the reference electrode.

In Example 235, the subject matter of any one or more of Examples 189-234 optionally includes and further comprising: correlating the difference in the measured capacitance between the test electrode and the reference electrode to a concentration of the predetermined biological molecule.

In Example 236, the subject matter of any one or more of Examples 189-235 optionally includes wherein the method includes measuring the property with an electrode array, wherein the electrode array comprises: a test electrode; a reference electrode; and a control electrode.

In Example 237, the subject matter of any one or more of Examples 189-236 optionally includes wherein the test electrode is functionalized with a recognition molecule.

In Example 238, the subject matter of any one or more of Examples 189-237 optionally includes wherein the test electrode is functionalized with a second recognition molecule.

In Example 239, the subject matter of any one or more of Examples 189-238 optionally includes wherein the recognition molecule is an antibody adapted to bind to a predetermined biological molecule.

In Example 240, the subject matter of any one or more of Examples 189-239 optionally includes wherein the second recognition molecule is a second antibody adapted to bind to a second preselected molecule of the biological sample.

In Example 241, the subject matter of any one or more of Examples 189-240 optionally includes wherein the reference electrode is free of any recognition molecules.

In Example 242, the subject matter of any one or more of Examples 189-241 optionally includes wherein the control electrode is functionalized with a blocking layer.

In Example 243, the subject matter of any one or more of Examples 189-242 optionally includes wherein the blocking layer is free of any bound preselected molecules.

In Example 244, the subject matter of any one or more of Examples 189-243 optionally includes wherein the blocking layer comprises a VSA protein.

In Example 245, the subject matter of any one or more of Examples 189-244 optionally includes and further comprising: supplying the biological sample from the sample collection probe to an absorbent sample pad; and supplying the biological sample from the sample pad to a conjugate pad, wherein the conjugate pad comprises a plurality of nanoparticles configured to bind to a predetermined biological molecule of the biological sample.

In Example 246, the subject matter of any one or more of Examples 189-245 optionally includes and further comprising: supplying the biological sample from the conjugate pad to a lateral flow strip.

In Example 247, the subject matter of any one or more of Examples 189-246 optionally includes wherein the lateral flow strip comprises: a test line adapted to attach to the plurality of nanoparticles bound to the preselected component of the biological sample; and a control line adapted to attach to nanoparticles that are free of the biological sample.

In Example 248, the subject matter of any one or more of Examples 189-247 optionally includes wherein the nanoparticles comprise antibodies.

In Example 249, the subject matter of any one or more of Examples 189-248 optionally includes wherein the test line comprises an activated surface.

In Example 250, the subject matter of any one or more of Examples 189-249 optionally includes wherein the activated surface comprises an antibody.

In Example 251, the subject matter of any one or more of Examples 189-250 optionally includes wherein the antibody is adapted to bind to a predetermined biological molecule.

In Example 252, the subject matter of any one or more of Examples 189-251 optionally includes and further comprising: supplying the biological sample from the conjugate pad to a second lateral flow strip.

In Example 253, the subject matter of any one or more of Examples 189-252 optionally includes and further comprising: contacting the test line with light; and measuring light emitted from the nanoparticles with a photodiode.

In Example 254, the subject matter of any one or more of Examples 189-253 optionally includes and further comprising: contacting the control line with light; and measuring light emitted from the nanoparticles with a photodiode.

In Example 255, the subject matter of any one or more of Examples 189-254 optionally includes and further comprising: lowering a pressure of an interior of the device to a pressure below atmospheric pressure.

In Example 256, the subject matter of any one or more of Examples 189-255 optionally includes and further comprising: powering a micropump to expel gas from the interior of the device.

In Example 257, the subject matter of any one or more of Examples 189-256 optionally includes wherein the micropump is powered by a dynamo adapted to generate electrical power and deliver the electrical power to the micropump.

In Example 258, the subject matter of any one or more of Examples 189-257 optionally includes wherein the dynamo comprises: a body; a coil of wires disposed along an interior surface of the body and defining a void; and a magnet disposed within the void.

In Example 259, the subject matter of any one or more of Examples 189-258 optionally includes and further comprising: attaching a dial to the magnet and extending outside of the body.

In Example 260, the subject matter of any one or more of Examples 189-259 optionally includes wherein the magnet is adapted to rotate in response to rotation of the dial.

In Example 261, the subject matter of any one or more of Examples 189-260 optionally includes wherein the micropump is powered by a capacitor.

In Example 262, the subject matter of any one or more of Examples 189-261 optionally includes and further comprising: charging the capacitor by collecting light with a solar panel.

In Example 263, the subject matter of any one or more of Examples 189-262 optionally includes and further comprising: charging the capacitor by striking a piezoelectric element.

In Example 264, the subject matter of any one or more of Examples 189-263 optionally includes wherein lowering the pressure comprises: depressing a flexible membrane to expel air from a one-way vent.

In Example 265, the subject matter of any one or more of Examples 189-264 optionally includes wherein the assay unit comprises a reagent cartridge.

In Example 266, the subject matter of any one or more of Examples 189-265 optionally includes and further comprising: supplying at least a portion of the biological sample from the sample collection probe to a sample channel; supplying the portion of the biological sample from the sample channel to a reagent well; and contacting the portion of the biological sample with a reagent in the reagent well.

In Example 267, the subject matter of any one or more of Examples 189-266 optionally includes wherein the reagent well comprises the reagent for processing a predetermined biomolecule.

In Example 268, the subject matter of any one or more of Examples 189-267 optionally includes wherein the reagent is an antibody.

In Example 269, the subject matter of any one or more of Examples 189-268 optionally includes and further comprising: supplying a second portion of the biological sample from the sample collection probe to a second sample channel; supplying the second portion of the biological sample from the second sample channel to a second reagent well; and contacting the second portion of the biological sample with a second reagent in the second reagent well.

In Example 270, the subject matter of any one or more of Examples 189-269 optionally includes and further comprising: measuring at least one of a temperature inside the cartridge, a humidity inside the cartridge, and a pressure inside the cartridge.

In Example 271, the subject matter of any one or more of Examples 189-270 optionally includes and further comprising: sensing at least one of contact between the cartridge and the housing, and leakage of the biological sample or reagent from the cartridge.

In Example 272, the subject matter of any one or more of Examples 189-271 optionally includes and further comprising: supplying the biological sample to a processing well connected to the reagent well by a reagent well exit channel; and supplying the biological sample to a detection well aligned with a detection system.

In Example 273, the subject matter of any one or more of Examples 189-272 optionally includes wherein the first reagent well and the second reagent well contain different reagents.

In Example 274, the subject matter of any one or more of Examples 189-273 optionally includes wherein at least one of the first reagent and the second reagent is stored in the reagent well in a lyophilized form.

In Example 275, the subject matter of any one or more of Examples 189-274 optionally includes and further comprising: hydrating the reagent with the biological sample.

In Example 276, the subject matter of any one or more of Examples 189-275 optionally includes and further comprising: heating the cartridge to a temperature sufficient to evaporate a component of at least one of the biological sample and the reagent; and expelling vapors of the evaporated component from the device through a vent.

In Example 277, the subject matter of any one or more of Examples 189-276 optionally includes wherein the cartridge is heated by a metallic layer attached to the device and adapted to contact the wearer.

In Example 278, the subject matter of any one or more of Examples 189-277 optionally includes wherein the metallic layer is adapted to absorb heat from the wearer.

In Example 279, the subject matter of any one or more of Examples 189-278 optionally includes and further comprising: heating the metallic layer with a microheater.

1-64. (canceled)

65. A wearable assay system comprising:
a housing comprising:
a first external surface adapted to contact a wearer; and
a void defined by a portion of the first external surface;
a sample collection probe positioned near the void and attached to the first external surface and adapted to collect a biological sample from the wearer;
an assay unit adapted to receive the biological sample from the sample collection probe;
an actuation system adapted to position the assay unit in contact with the sample collection probe; and
a detection system adapted to detect a property of the biological sample.

66. The wearable assay system of claim 65, and further comprising:
a second assay unit adapted to receive the biological sample from the sample collection probe.

67. The wearable assay system of claim 65, wherein the assay unit comprises:
an array of lateral flow strips adapted to receive the biological sample from the sample collection probe, wherein the array of lateral flow strips comprises:
a filter pad adapted to contact the sample collection probe;
a first lateral flow strip attached to a first location of the filter pad; and
a second lateral flow strip attached to a second location of the filter pad;
an actuation system adapted to position the assay unit in contact with the sample collection probe; and
a removable blocking pad disposed within the filter pad.

68. The wearable assay system of claim 67, wherein the blocking pad is disposed between the first lateral flow strip and the second lateral flow strip.

69. The wearable assay system of claim 65, further comprising a pumping system adapted to pump the biological sample through the wearable assay system.

70. A wearable assay system comprising:
a housing comprising:
a first external surface adapted to contact a wearer; and
a void defined by a portion of the first external surface;
a sample collection probe positioned near the void and attached to the first external surface and adapted to collect a biological sample from the wearer;
an assay unit comprising:
an array of lateral flow strips adapted to receive the biological sample from the sample collection probe; and
an actuation system adapted to position the assay unit in contact with the sample collection probe.

71. The wearable assay system of claim 70, wherein the array of lateral flow strips comprises:
a filter pad adapted to contact the sample collection probe;
a first lateral flow strip attached to a first location of the filter pad; and
a second lateral flow strip attached to a second location of the filter pad.

72. The wearable assay system of claim 71, wherein the first lateral flow strip is joined to the filter pad at a first end and to a first wick pad at a second end.

73. The wearable assay system of claim 71, wherein the second lateral flow strip is joined to the filter pad at a first end and to a second wick pad at a second end.

74. The wearable assay system of claim 71, wherein the first lateral flow strip and the second lateral flow strip are the same size.

75. A wearable assay system comprising:
a housing comprising:
a first external surface adapted to contact a wearer; and
a void defined by a portion of the first external surface;
a sample collection probe positioned near the void and attached to the first external surface and adapted to collect a biological sample from the wearer; and
an assay unit adapted to receive the biological sample from the sample collection probe, the assay unit comprising:
an electrode array comprising:
a test electrode;
a reference electrode; and
a control electrode.

76. The wearable assay system of claim 75, wherein the test electrode is functionalized with a recognition molecule.

77. The wearable assay system of claim 76, wherein the recognition molecule is an antibody adapted to bind to a preselected molecule of the biological sample.

78. The wearable assay system of claim 75, wherein an output voltage of the electrode array in the presence of the biological sample is different than an output voltage of the electrode array in the absence of the biological sample.

79. The wearable assay system of claim 75, wherein the electrode array further comprises:
a second test electrode;
a second reference electrode; and
a second control electrode, wherein the first test electrode, second test electrode, first control electrode, and second control electrode are substantially the same size.

80. The wearable assay system of claim 75, wherein the reference electrode is larger than at least one of the test electrode and the control electrode.

81. The wearable assay system of claim 75, wherein the test electrode and the reference electrode are formed from the same material.

82. A wearable assay system comprising:
a housing comprising:
a first external surface adapted to contact a wearer; and
a void defined by a portion of the first external surface;
a sample collection probe positioned near the void and attached to the first external surface and adapted to collect a biological sample from the wearer; and
an assay unit adapted to receive the biological sample from the sample collection probe, the assay unit comprising:
a reagent cartridge comprising:
a sample channel connected to the sample collection probe;
a reagent well connected to the sample channel; and
a reagent well exit channel connected to the reagent well.

83. The wearable assay system of claim 82, wherein the cartridge further comprises:
a second sample channel connected to the sample collection probe;
a second reagent well connected to the sample channel; and
a second reagent well exit channel connected to a second reagent well.

84. The wearable assay system of claim 82, wherein the cartridge further comprises:
a sensor adapted to measure at least one of a temperature inside the cartridge, a humidity inside the cartridge, and a pressure inside the cartridge.

85. The wearable assay system of claim 83, wherein the first reagent well and the second reagent well contain different reagents.

86. The wearable assay system of claim 82, and further comprising:
a thermally conductive metallic layer attached to the first external surface of the housing; and
a vent defined by a second external surface of the housing.

87. A method of performing an assay on a wearable device, the method comprising:
contacting a sample collection probe with a layer of skin of a wearer;
collecting a biological sample from the wearer with the sample collection probe;
pumping the biological sample from the sample collection probe to a first assay unit; and
detecting a property of the biological sample.

88. The method of claim 87, and further comprising:
pumping the biological sample to a second assay unit.

89. The method of claim 88, wherein the first assay unit and the second assay unit are selectively placed in fluid communication with the sample collection probe by an actuation system.
